(19) United States
(12) Patent Application Publication
(10) Pub. No.: US 2025/0090508 A1
(43) Pub. Date: Mar. 20, 2025

(54) METHODS OF REDUCING ITCH USING TOPICAL ROFLUMILAST COMPOSITIONS

(71) Applicant: ARCUTIS BIOTHERAPEUTICS, INC., Westlake Village, CA (US)

(72) Inventors: David Reuben BERK, Westlake Village, CA (US); Patrick Burnett, Westlake Village, CA (US); David Chu, Westlake Village, CA (US); Howard Welgus, Ballwin, MO (US)

(73) Assignee: ARCUTIS BIOTHERAPEUTICS, INC., Westlake Village, CA (US)

(21) Appl. No.: 18/884,931

(22) Filed: Sep. 13, 2024

Related U.S. Application Data
(60) Provisional application No. 63/538,667, filed on Sep. 15, 2023.
(60) Provisional application No. 63/543,858, filed on Oct. 12, 2023.

(51) Int. Cl. A61K 31/44; A61K 9/00; A61K 9/06; A61K 47/10; A61K 47/24; A61P 17/00; A61P 17/04

(52) U.S. Cl. CPC A61K 31/44; A61K 9/0014; A61K 9/06; A61K 47/10; A61K 47/24; A61P 17/00; A61P 17/04

(57) ABSTRACT

Methods of treating a skin disorder or condition, including psoriasis, atopic dermatitis, and seborrheic dermatitis, in a patient by topically administering to the patient a pharmaceutical composition comprising roflumilast. The methods rapidly (e.g., within 24 or 48 hours) reduce itch experienced by a patient, for example itch as measured by the Worst Itch Numerical Rating Scale (WI-NRS). The method can also reduce itch as measured by the WI-NRS by 4 or more points.

### CROSS REFERENCE TO RELATED APPLICATIONS

This application claims the benefit of U.S. Provisional Application No. 63/538,667 filed Sep. 15, 2023 and U.S. Provisional Application No. 63/543,858 filed on Oct. 12, 2023, the disclosures of which are incorporated herein in their entirety by reference.

### FIELD OF THE INVENTION

The subject matter disclosed herein generally relates to methods of treating a skin disorder or condition in a patient by topically administering to the patient a pharmaceutical composition comprising roflumilast. The methods can rapidly (e.g., within 24 or 48 hours) reduce itch experienced by a patient, for example itch as measured by the Worst Itch Numerical Rating Scale (WI-NRS).

### BACKGROUND OF THE INVENTION

Roflumilast is an inhibitor of phosphodiesterase (PDE) type 4. Oral pharmaceutical compositions of roflumilast are currently marketed under the tradenames Daliresp® (in the United States) and Daxas® (in Europe). Oral pharmaceutical compositions of roflumilast are indicated as a treatment to reduce the risk of chronic obstructive pulmonary disease (COPD) exacerbations in patients with severe COPD associated with chronic bronchitis and a history of exacerbations. Topical pharmaceutical compositions of roflumilast are currently marketed under the tradename Zoryve®. Topical roflumilast compositions are indicated for the treatment of plaque psoriasis-including intertriginous areas.

### SUMMARY OF THE INVENTION

The present invention relates to methods of treating a skin disorder or condition in a patient by topically administering to the patient a pharmaceutical composition comprising roflumilast. The methods comprise administering to the patient a pharmaceutical composition comprising roflumilast. In certain embodiments, the patient is suffering from a skin disorder or condition selected from the group consisting of psoriasis, atopic dermatitis, and seborrheic dermatitis. The methods can rapidly (e.g., within 24 or 48 hours) reduce itch experienced by a patient, for example itch as measured by the Worst Itch Numerical Rating Scale (WI-NRS).

In certain embodiments, a method of treating a skin disorder or condition in a patient is provided. In certain embodiments, the methods are used to treat psoriasis, atopic dermatitis, or seborrheic dermatitis. In certain embodiments, the method includes topically administering to the patient a pharmaceutical composition comprising roflumilast. In certain embodiments, the methods rapidly (e.g., within 24 or 48 hours) reduce itch experienced by a patient, for example itch as measured by the Worst Itch Numerical Rating Scale (WI-NRS). In certain embodiments, the pharmaceutical composition comprising roflumilast reduces itch 24 hours after topical application in a patient with atopic dermatitis. In certain embodiments, topical application of the roflumilast composition reduces the severity of itch. In certain embodiments, topical application of the roflumilast composition reduces bother caused by itch.

In certain embodiments, a method for reducing itch in a patient suffering from a skin disorder or condition is provided. The method includes topically administering to the patient a pharmaceutical composition comprising roflumilast. The method rapidly and significantly reduces itch in a patient. In certain embodiments, the method reduces the patient's itch in less than or equal to 24 hours after topical administration of the pharmaceutical composition comprising roflumilast. The method reduces itch as measured by the WI-NRS by 1 or more points, by 2 or more points, by 3 or more points, by 4 or more points, by 5 or more points, or by 6 or more points. In certain embodiments, the method reduces itch as measured by the WI-NRS by 4 or more points within four weeks following administration of the pharmaceutical composition comprising roflumilast. In certain embodiments, the method reduces itch as measured by the WI-NRS by 4 or more points within four weeks following once daily administration of the pharmaceutical composition comprising roflumilast.

In certain embodiments, a method for reducing itch in a patient between the ages of 2 and 5 years old and suffering from a skin disorder or condition is provided. The method includes topically administering to the patient a pharmaceutical composition comprising roflumilast. The method rapidly and significantly reduces itch in a patient. In certain embodiments, the method reduces the patient's itch in less than or equal to 24 hours after topical administration of the pharmaceutical composition comprising roflumilast. The method reduces itch as measured by the WI-NRS by 1 or more points, by 2 or more points, by 3 or more points, by 4 or more points, by 5 or more points, or by 6 or more points. In certain embodiments, the method reduces itch as measured by the WI-NRS by 4 or more points within four weeks following administration of the pharmaceutical composition comprising roflumilast. In certain embodiments, the method reduces itch as measured by the WI-NRS by 4 or more points within four weeks following once daily administration of the pharmaceutical composition comprising roflumilast.

### BRIEF DESCRIPTION OF THE DRAWINGS

The accompanying drawings, which are incorporated herein and form part of the disclosure, help illustrate various embodiments of the present invention and, together with the description, further serve to describe the invention to enable a person skilled in the pertinent art to make and use the embodiments disclosed herein. The error bars in the drawings are standard deviation values.

FIG. 1 provides a line plot of the mean daily WI-NRS score over time for two treatment groups (roflumilast cream 0.15% versus vehicle) from Study 1 as described in Example 6.

FIG. 2 provides a line plot of the mean daily WI-NRS score over time for two treatment groups (roflumilast cream 0.15% versus vehicle) from Study 2 as described in Example 6.

FIG. 3 provides a line plot of the percent change from baseline in daily WI-NRS over time for two treatment groups (roflumilast cream 0.15% versus vehicle) from Study 1 as described in Example 6.

FIG. 4 provides a line plot of the percent change from baseline in daily WI-NRS over time for two treatment groups (roflumilast cream 0.15% versus vehicle) from Study 2 as described in Example 6.

FIG. 5 provides a line plot of the mean change from baseline in WI-NRS score from Study 1 as described in Example 6.

FIG. 6 provides a line plot of the mean change from baseline in WI-NRS score from Study 2 as described in Example 6.

FIG. 7 provides a line plot of the mean daily WI-NRS score over time by treatment group (roflumilast cream 0.05% versus vehicle) as described in Example 7.

FIG. 8 provides a line plot of the percent change from baseline in daily WI-NRS over time for two treatment groups (roflumilast cream 0.05% versus vehicle) as described in Example 7.

FIG. 9 provides a line plot of mean change from baseline in WI-NRS score over time by treatment group (roflumilast cream 0.05% versus vehicle) as described in Example 7.

FIG. 10 shows the proportion of patients aged ≥12 years with baseline Worst Itch-Numeric Rating Scale (WI-NRS)≥4 who achieved >4-point improvement in WI-NRS from the trial baseline over time, including both patients treated once daily and twice weekly.

### DETAILED DESCRIPTION OF THE INVENTION

Before the present invention is described in detail below, it is to be understood that this invention is not limited to the particular methodology, protocols, and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Note that as used herein, the singular forms “a,” “an,” and “the” include plural referents unless the context clearly dictates otherwise. Thus, for example, “active ingredient” includes a single ingredient and two or more different ingredients.

The term “about” when used in connection with a numerical value is meant to encompass numerical values within a range having a lower limit that is 5% smaller than the indicated numerical value and having an upper limit that is 5% larger than the indicated numerical value.

The term “effective” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances.

The term “pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “roflumilast” as used in this application refers to roflumilast and its salts unless specified otherwise or unless it is clear in context that reference is to roflumilast itself.

As used herein, the terms “subject” or “patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration.

The term “topical” with respect to administration of a drug or composition refers to the application of such drug or composition to the epithelial surface outside the body, including the skin or cornea. For this application, localized delivery to the mucosa inside of a body opening mucosal surface, such as the mouth, vagina, or rectum, is considered a topical application.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The abbreviation “w/v” represents the relative concentration of the components in the composition as “weight to volume.”

The abbreviation “w/w” represents the relative concentration of the components in the composition as “weight to weight” (i.e., percentage refers to percentage of total weight), rather than based on volume or other quantities.

The present invention relates to methods of treating a patient by administering a pharmaceutical composition of roflumilast to the patient. In certain embodiments, the method comprises topically administering to a patient a pharmaceutical composition comprising a therapeutically effective amount of the phosphodiesterase-4 inhibitor, roflumilast or a pharmaceutically acceptable salt thereof.

Roflumilast is a compound of the formula (I):

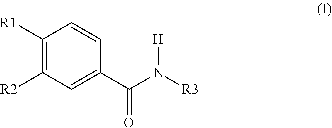

wherein R1 is difluoromethoxy, R2 is cyclopropylmethoxy and R3 is 3,5-dichloropyrid-4-yl.

Roflumilast has the chemical name N-(3,5-dichloropyrid-4-yl)-3-cyclopropylmethoxy-4-difluoromethoxybenzamide. Roflumilast and its synthesis are described in U.S. Pat. No. 5,712,298, which is incorporated herein by reference. The pharmaceutical composition described herein can include roflumilast as a free base or a pharmaceutically acceptable salt. Exemplary salts of roflumilast are salt described in paragraphs and of U.S. Patent Application Publication No. US 2006/0084684, the disclosure of which is incorporated herein by reference.

### Methods for Reducing Itch

In certain embodiments, a method of treating a skin disorder or condition in a patient is provided. The method includes topically administering to the patient a pharmaceutical composition comprising roflumilast. The methods can rapidly (e.g., within 24 or 48 hours) reduce itch experienced by a patient, for example itch as measured by the Worst Itch Numerical Rating Scale (WI-NRS).

The WI-NRS is a simple, single item to assess the patient-reported severity of this symptom at its highest intensity during the previous 24-hour period. See, e.g., Newton 2019. The WI-NRS is determined by the subject's recording of daily assessment of worst itch over the past 24 hours. The scale is from ‘0 to 10’ (“no itch” to “worst imaginable itch” or “worst itch imaginable”). Patients are asked to “[p]lease rate your itching severity by circling the number that best describes your worst level of itching in the past 24 hours.” The WI-NRS score can be determined by a parent/caregiver if the patient is a pediatric patient.

In certain embodiments, the patient is suffering from scalp psoriasis and the method can rapidly (e.g., within 24 or 48 hours) reduce scalp itch experienced by a patient, for example scalp itch as measured by the Scalp Itch Numeric Rating Scale (SI-NRS). The SI-NRS is assessed in a similar manner to WI-NRS (discussed above), but is used to assess scalp itch.

In certain embodiments, a method for reducing itch in a patient suffering from a skin disorder or condition is provided. In certain embodiments, the methods are used to treat itch in patients suffering from psoriasis, atopic dermatitis, or seborrheic dermatitis. The method includes topically administering to the patient a pharmaceutical composition comprising roflumilast. The method can rapidly and significantly reduce itch in a patient. In certain embodiments, the method reduces the patient's itch in less than or equal to 24 hours after topical administration of the pharmaceutical composition comprising roflumilast. In certain embodiments, the method reduces the patient's itch in less than or equal to 48 hours after topical administration of the pharmaceutical composition comprising roflumilast. The method can reduce itch as measured by the WI-NRS by 1 or more points, by 2 or more points, by 3 or more points, by 4 or more points, by 5 or more points, or by 6 or more points. In certain embodiments, the method reduces itch as measured by the WI-NRS by 4 or more points within 24 hours following administration of the pharmaceutical composition comprising roflumilast. In certain embodiments, the method reduces itch as measured by the WI-NRS by 4 or more points within 48 hours following administration of the pharmaceutical composition comprising roflumilast. In certain embodiments, the method reduces itch as measured by the WI-NRS by 4 or more points within one week following once daily administration of the pharmaceutical composition comprising roflumilast. In certain embodiments, the method reduces itch as measured by the WI-NRS by 4 or more points within two weeks following once daily administration of the pharmaceutical composition comprising roflumilast. In certain embodiments, the method reduces itch as measured by the WI-NRS by 4 or more points within four weeks following once daily administration of the pharmaceutical composition comprising roflumilast.

In certain embodiments, the patient is a patient suffering from atopic dermatitis. The patient can be selected based on a diagnosis of mild to moderate atopic dermatitis according to the criteria of Hanifin and Rajka (1980). In certain embodiments, subjects are selected based on having at least three of the four basic features of: (1) pruritus; (2) typical morphology and distribution (flexural lichenification in adults and facial and extensor eruptions in infants and children); (3) chronic or chronically relapsing dermatitis; or (4) personal or family history of atopy. In certain embodiments, the patient has a history of atopic dermatitis for at least 3 months in patients aged 6-17 years old or 6 months in patients aged 18 or older. In certain embodiments, the patient is a patient between the ages of 2 to 5 years old. In certain embodiments, the patient has an Eczema Area and Severity Index (EASI) score of equal to or greater than 5 at baseline. EASI is evaluated for the entire body except the scalp, palms, and soles. In certain embodiments, the patient can have a validated Investigator Global Assessment Scale for Atopic Dermatitis (vIGA-AD) score of Mild (2) or Moderate (3) at baseline. vIGA-AD is evaluated for the entire body except the scalp, palms, and soles.

### Dosing Regimens

In certain embodiments, the pharmaceutical composition is administered as a regimen, such as at regular intervals. For example, a pharmaceutical composition can be administered once daily, twice daily, thrice daily, four times daily, once per week, twice per week, three times per week, or four times per week, weekly, or as needed (pro re nata or PRN). The pharmaceutical composition can be administered for a prescribed period of time. For example, a pharmaceutical composition can be administered for a period of about two days or more, or until an improvement in the condition or disease is observed. Exemplary periods of time for the treatment regimen include one week, two weeks, three weeks, one month, six weeks, two months, three months, four months, five months, six months, seven months, eight months, nine months, ten months, eleven months, one year, or two years. The pharmaceutical composition can be topically administered as an ongoing treatment with no end.

In certain embodiments, the methods comprise sequentially administering to the patient multiple doses of the pharmaceutical composition comprising roflumilast. In certain embodiments, the methods include sequentially administering to the patient initial doses of the pharmaceutical composition comprising roflumilast at a first interval, followed by one or more maintenance doses of the of the pharmaceutical composition comprising roflumilast at a second interval. In certain embodiments, the first interval is once a day. In certain embodiments, the second interval is twice a week. In certain embodiments, the roflumilast is administered topically once daily until the symptoms of the skin condition or disease have improved, then the patient switches to a maintenance dosing regimen of twice weekly administration. In certain embodiments, the roflumilast is administered topically once daily until the patient has no signs or symptoms of the skin condition or disease, then the patient switches to a maintenance dosing regimen of twice weekly administration.

In certain embodiments, a patient is treated by topically administering the patient a pharmaceutical roflumilast composition once a day for a period of time. In certain embodiments, the period of time is one week, two weeks, three weeks, four weeks, five weeks, six weeks, seven weeks, eight weeks or more. In certain embodiments, the patient is administered a pharmaceutical roflumilast composition once daily for four weeks. The patient is then administered the pharmaceutical roflumilast composition twice a week (B.I.W.) as a maintenance dosing regimen. In certain embodiments, the patient is treated by topically administering pharmaceutical roflumilast composition once a day for four weeks. Thereafter, the roflumilast is administered twice a week as long as the patient does not experience recurrence or worsening of symptoms.

In certain embodiments, the maintenance dosing regimen is given to a patient for one week, two weeks, three weeks, four weeks, five weeks, six weeks, seven weeks, eight weeks, three months, four months, five months, six months, seven months, eight months, nine months, ten months, up to a year or more. In certain embodiments, the pharmaceutical composition can be topically administered as an ongoing treatment with no end. The frequency of administration may also be adjusted during the course of treatment by a physician depending on the needs of the individual patient following clinical examination. For example, the patient may switch back to once daily administration from twice a week administration after a certain period of time, then return to twice a week administration.

In certain embodiments, a patient is treated by topically administering a roflumilast composition once a day until the patient's disease is under control. In certain embodiments, the patient's disease is under control when the patient no longer exhibits symptoms of the disease. The patient is then administered the roflumilast composition twice a week as a maintenance regimen. In certain embodiments, the maintenance dosing of the pharmaceutical roflumilast composition maintains the disease state (or lack thereof) in the patient.

In certain embodiments, the patient is administered a topical pharmaceutical roflumilast composition once a day until the patient achieves a validated Investigator Global Assessment Scale for Atopic Dermatitis (vIGA-AD) score of zero. After the patient achieves a vIGA-AD score of zero, the patient is then administered the pharmaceutical roflumilast composition twice a week as a maintenance dosing regimen. In certain embodiments, the maintenance dosing is able to maintain the patient's vIGA-AD score, wherein the vIGA-AD score is zero or one. In certain embodiments, the maintenance dosing is able to improve the patient's vIGA-AD score.

The vIGA-AD is a static evaluation of qualitative overall AD severity. This global assessment scale is an ordinal scale with five severity grades (reported only in integers of 0 to 4). Each grade is defined by a distinct and clinically relevant morphologic description that minimizes inter-observer variability (see Table 1). vIGA-AD is evaluated for the entire body except the scalp, palms, and soles.

TABLE 1
vIGA-AD Score.

| Score | Morphological Description |
|---|---|
| 0 - Clear | No inflammatory signs of atopic dermatitis (no erythema, no |
| | induration/papulation, no lichenification, no oozing/crusting). Post- |
| | inflammatory hyperpigmentation and/or hypopigmentation may be |
| | present. |
| 1 - Almost Clear | Barely perceptible erythema, barely perceptible induration/papulation, |
| | and/or minimal lichenification. No oozing or crusting. |
| 2 - Mild | Slight but definite erythema (pink), slight but definite |
| | induration/papulation, and/or slight but definite lichenification. No |
| | oozing or crusting. |
| 3 - Moderate | Clearly perceptible erythema (dull red), clearly perceptible |
| | induration/papulation, and/or clearly perceptible lichenification. |
| | Oozing and crusting may be present. |
| 4 - Severe | Marked erythema (deep or bright red), marked induration/papulation, |
| | and/or marked lichenification. Disease is widespread in extent. Oozing |
| | or crusting may be present. |

In certain embodiments, the two phase dosing regimen can improve and maintain efficacy over the course of administration. In certain embodiments, the two phase dosing regimen can achieve 2-grade improvement from baseline in vIGA-AD score. The two phase dosing regimens described herein can provide a patient with improved convenience, as the dosing regimens require less frequent administration compared to previous dosing regimens requiring daily administration while at the same time maintaining disease control. The ability to maintain a twice a week dosing schedule while maintaining disease control for a prolonged time despite low systemic exposure (sometimes below the limit of quantification) was surprising and unexpected.

### Skin Disorders and Conditions

The composition can be used in veterinary and in human medicine for the treatment and prevention of skin disorders and conditions, including but not limited to proliferative, inflammatory and allergic dermatoses such as psoriasis, scalp psoriasis, or inverse psoriasis, irritant and allergic contact eczema, hand eczema, atopic dermatitis, seborrheic dermatitis, lichen simplex, sunburn, aphthous ulcers, lichen planus, vitiligo, lichen sclerosis et atrophicus (especially of the female genitalia), pruritus in the genital or anal regions, alopecia areata, hypertrophic scars, discoid lupus erythematosus, follicular and extensive pyodermas, endogenous and exogenous acne, acne rosacea, pruritus of unknown origin, and neuralgia paresthetica. In certain embodiments, the methods are used to treat proliferative, inflammatory and allergic dermatoses such as psoriasis (vulgaris), eczema, acne, lichen simplex, sunburn, pruritus, alopecia areata, hypertrophic scars, discoid lupus erythematosus, and pyodermas. In preferred embodiments, the methods are used to treat psoriasis, atopic dermatitis, or seborrheic dermatitis. In certain embodiments, the method is used to treat prurigo nodularis, prurigo pigmentosa, or neurotic excoriations.

In certain embodiments, a method of treating a patient suffering from an epidermal disorder of persistent inflammation is provided. The method comprises topically administering to the patient a pharmaceutical composition comprising a therapeutically effective amount of roflumilast. In certain embodiments, the epidermal disorder of persistent inflammation is a cell kinetic and differentiation disorder. In certain embodiments, the epidermal disorder of persistent inflammation is selected from the group consisting of pityriasis rubra pilaris, pityriasis rosea, pityriasis lichenoides, notalgia paresthetica, lichen planus, lichen nitidus, lichen simplex chronicus, hyperkeratosis lenticularis perstans (Flegel's disease), inherited keratodermas of palms and soles, granuloma annulare, Sweet's syndrome, pyoderma gangrenosum, and urticaria, including chronic idiopathic urticaria, physical urticaria, and other forms of acquired urticaria.

In certain embodiments, a method of treating a patient suffering from an epidermal disorder of persistent inflammation is provided wherein the disorder is an altered reactivity disorder. In certain embodiments, the epidermal disorder of persistent inflammation is selected from the group consisting of perioral dermatitis, allergic contact dermatitis, irritant contact dermatitis, intertrigo, nummular eczematous dermatitis, dyshidrotic eczema, hand eczema, and vesicular palmoplantar eczema.

In certain embodiments, a method of treating a patient suffering from a disorder of lips, oral, or vaginal mucosa is provided. The method comprises topically administering to the patient a pharmaceutical composition comprising a therapeutically effective amount of roflumilast. In certain embodiments, the disorder of lips, oral, or vaginal mucosa is selected from the group consisting of actinic cheilitis, aphthous ulcers, vulvar aphthae, oral lichen planus, geographic tongue, lichen sclerosis et atrophicus, especially lichen sclerosis et atrophicus of the female genitalia, feminine itch, Behcet's disease, and linear IgA.

In certain embodiments, a method of treating a patient suffering from an epidermal disorder of cohesion is provided. The method comprises topically administering to the patient a pharmaceutical composition comprising a therapeutically effective amount of roflumilast. In certain embodiments, the disorder of epidermal disorder of cohesion is a vesiculobullous disorder selected from the group consisting of erythema multiforme, pemphigus, bullous pemphigoid, linear IgA dermatosis, herpes gestationis, dermatitis herpetiformis, Hailey-Hailey disease, pustular palmoplantar psoriasis, herpes simplex virus I and II, eczema herpeticum, epidermolysis bullosa acquisita, Behcet's disease, and Darier's disease.

In certain embodiments, a method of treating a patient suffering from a disorder of epidermal appendages is provided. The method comprises topically administering to the patient a pharmaceutical composition comprising a therapeutically effective amount of roflumilast. In certain embodiments, the disorder of epidermal appendages is selected from the group consisting of acne vulgaris, frontal fibrosing alopecia, alopecia areata, rosacea, and hidradenitis suppurativa.

In certain embodiments, a method of treating a patient suffering from hypomelanoses or hypermelanoses is provided. The method comprises topically administering to the patient a pharmaceutical composition comprising a therapeutically effective amount of roflumilast. In certain embodiments, the hypomelanoses or hypermelanoses is vitiligo, dyschromia, pityriasis alba, post-inflammatory hypo-pigmentation, or post-inflammatory hyper-pigmentation.

In certain embodiments, a method of treating a patient suffering from a cutaneous T-cell or B-cell lymphoma is provided. The method comprises topically administering to the patient a pharmaceutical composition comprising a therapeutically effective amount of roflumilast. In certain embodiments, the cutaneous lymphoma is selected from the group consisting of lymphomatous papulosis, cutaneous T-cell lymphoma (CTCL), and non-mycosis fungoides cutaneous T-cell or B-cell lymphoma.

In certain embodiments, a method of treating a patient suffering from a skin manifestation of a rheumatologic disease is provided. The method comprises topically administering to the patient a pharmaceutical composition comprising a therapeutically effective amount of roflumilast. In certain embodiments, the skin manifestation of a rheumatologic disease is selected from the group consisting of cutaneous lupus erythematosus, discoid lupus, dermatomyositis, sarcoidosis, chronic idiopathic pruritus, cutaneous vasculitis, and scleroderma/morphea.

In certain embodiments, a method of treating a patient suffering from a skin manifestation related to an oncology disorder is provided. The method comprises topically administering to the patient a pharmaceutical composition comprising a therapeutically effective amount of roflumilast. In certain embodiments, the skin manifestation of an oncology disorder is selected from the group consisting of adverse skin reactions due to epidermal growth factor receptor (EGFR) inhibitors, adverse skin reactions due to cytotoxic T-lymphocyte antigen-4 (CTLA-4) inhibitors, adverse skin reactions due to programmed cell death-1 (PD-1) inhibitors or other checkpoint inhibitors, and adverse skin reactions due to BRAF inhibitors.

### Amount of Roflumilast Administered

Each dose of roflumilast administered to the patient over the course of the treatment regimen may contain the same, or substantially the same, amount of roflumilast. Alternatively, the amount of roflumilast contained within the individual doses may vary over the course of the treatment regimen. For example, the concentration of roflumilast contained within the individual pharmaceutical composition administered can increase over time (e.g., each subsequent dose can contain more roflumilast than the last), decrease over time (e.g., each subsequent dose can contain less roflumilast than the last), initially increase then decrease, initially decrease then increase, or remain the same throughout the course of the treatment regimen.

The amount of roflumilast administered to the patient in each dose can be a therapeutically effective amount. In certain embodiments, the pharmaceutical composition can comprise roflumilast in a range from about 0.01% to about 3.0%, or from about 0.01% to about 2.0%, or from about 0.01% to about 1.0%, or from about 0.01% to about 0.3%, or from about 0.05% to about 0.3%, or from about 0.15% to about 0.3%. For example, the pharmaceutical composition can comprise any of the following concentration of roflumilast: 0.05%, 0.15%, and 0.3%. In certain embodiments, wherein the patient is a child between the ages of 2 and 5 years old, the pharmaceutical composition can comprise 0.05% roflumilast.

### Pharmaceutical Compositions

The present invention includes methods of treatment wherein the roflumilast administered to the patient is contained within a pharmaceutical composition. The pharmaceutical composition can comprise roflumilast and at least one inactive ingredient, such as a pharmaceutically acceptable carrier. In preferred embodiments, the pharmaceutical composition is a topical pharmaceutical composition comprising roflumilast.

Topical pharmaceutical compositions of roflumilast are described in U.S. Pat. Nos. 9,895,359, 11,534,493, U.S. application Ser. No. 17/821,051, and U.S. application Ser. No. 17/887,798, which are incorporated by reference herein. In certain embodiments, the pharmaceutical composition is one of the compositions disclosed in one of U.S. Pat. Nos. 9,895,359, 11,534,493, U.S. application Ser. No. 17/821,051, or U.S. application Ser. No. 17/887,798.

In certain embodiments, the pharmaceutical compositions disclosed herein comprises diethylene glycol monoethyl ether and water. Diethylene glycol monoethyl ether is also known as 2-(2-ethoxyethoxy) ethanol, or as DEGEE, and is marketed under the several tradenames, including Transcutol® (Gattefosse Corporation, Paramus, NJ), Carbitol™ (The Dow Chemical Company, Midland, MI), Dioxitol® (Shell Oil Company, Houston, TX), and Poly-Solv DM (Monument Chemical, Houston, TX).

The concentration of the diethylene glycol monoethyl ether in the formulation is that which is sufficient to dissolve the active pharmaceutical ingredient and to provide antimicrobial properties. In certain embodiments, the pharmaceutical composition comprises greater than 25% w/w of diethylene glycol monoethyl ether, which is sufficient to provide antimicrobial properties. In certain embodiments, the amount of diethylene glycol monoethyl ether can range from about 20% w/w to about 40% w/w, about 20% w/w to about 35% w/w, about 25% w/w to about 35% w/w, about 20% w/w to about 30% w/w, or about 25% w/w to about 30% w/w. For example, the pharmaceutical composition comprises any of the following w/w percents of diethylene glycol monoethyl ether: 20%, 21%, 22%, 23%, 24%, 25%, 26%, 27%, 28%, 29%, 30%, 31%, 32%, 33%, 34%, 35%, 36%, 37%, 38%, 39%, 40%, etc.

In certain embodiments, the pharmaceutical composition further comprises an emulsifier blend comprising cetearyl alcohol (CAS 67762 30 0), dicetyl phosphate (CAS 2197 63 9), and ceteth-10 phosphate (CAS 50643-20-4). An emulsifier blend comprising cetearyl alcohol, dicetyl phosphate and ceteth-10 phosphate is manufactured by Croda under the tradename Crodafos™ CES. Crodafos™ CES PHARMA is manufactured using the same starting materials and process, but undergoes enhanced quality control and release testing and uses the nomenclature cetearyl alcohol, cetearyl phosphate and ceteareth-10 phosphate in keeping with standard practice for naming pharmaceutical excipients. This commercially available emulsifier blend is a self-emulsifying wax that is predominately the waxy material cetearyl alcohol (which is a mixture of cetyl alcohol (C₁₆H₃₄O) and stearyl alcohol (C₁₈H₃₈O)) combined with 10-20% dicetyl phosphate (cetearyl phosphate) and 10-20% ceteth-10 phosphate (ceteareth-10 phosphate). Self-emulsifying waxes form an emulsion when blended with water. When Crodafos™ CES is added to water it spontaneously forms an emulsion having a pH of about 3. Agents which adjust the pH, such as sodium hydroxide solution, can be added to increase the pH to the desired value.

In certain embodiments, the amount of the emulsifier blend of cetearyl alcohol, dicetyl phosphate, and ceteth-10 phosphate can range from about 5% w/w to about 20% w/w, about 6% w/w to about 20% w/w, about 8% w/w to about 20% w/w, about 6% w/w to about 15% w/w, or about 8% w/w to about 15% w/w. For example, the pharmaceutical composition comprises any of the following w/w percents of the emulsifier blend of cetearyl alcohol, dicetyl phosphate, and ceteth-10 phosphate: 5%, 6%, 7%, 8%, 9%, 10%, 11%, 12% 13%, 14%, 15%, 16%, 17%, 18%, 19%, 20%, etc.

The topical formulations disclosed herein can be emulsions, suspensions, sprays, oils, ointments, fatty ointments, creams, lotions, pastes, gels, or foams. In preferred embodiments, the pharmaceutical compositions can be formulated as an emulsion in the form of a cream or foam.

For example, the pharmaceutical composition can be formulated as an emulsion comprising a solvent, water, an emulsifier, and a moisturizer or as one of the following forms:

An oil-in-water emulsion: The pharmaceutical composition may be an emulsion comprising a discrete phase of a hydrophobic component and a continuous aqueous phase that includes water and optionally one or more polar hydrophilic excipients as well as solvents, co-solvents, salts, surfactants, emulsifiers, and other components. These emulsions may include water-soluble or water-swellable polymers that help to stabilize the emulsion.

A water-in-oil emulsion: The pharmaceutical composition may be an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) to help to stabilize the emulsion.

A microemulsion: The pharmaceutical composition may be a clear, thermodynamically stable isotropic liquid system that contains oil, water and surfactants, frequently in combination with a cosurfactant. Microemulsions may be water continuous, oil continuous or bicontinuous mixtures. The pharmaceutical composition may optionally also contain water up to 60% by weight. Higher levels may be suitable in some compositions.

A nanoemulsion: The pharmaceutical composition may be an isotropic dispersed system that contains water, oil, and an emulsifier. The system may be an oily system dispersed in an aqueous system, or an aqueous system dispersed in an oily system forming droplets or oily phases of nanometric sizes. Nanoemulsions often have higher loading capacity for lipophilic active ingredients than microemulsions. Hydrophobic and hydrophilic active ingredients can also be formulated in nanoemulsion. Nanoemulsions may be formed by any suitable method known in the art, including high-pressure homogenization, microfluidization, and phase-inversion temperature.

Thickened aqueous gels: The pharmaceutical composition may include an aqueous phase which has been thickened by suitable natural, modified natural, or synthetic thickeners such as described below. Alternatively, the thickened aqueous gels can be thickened using suitable polyethoxylate alky chain surfactants or other nonionic, cationic, or anionic systems.

Thickened hydroalcoholic gels: The pharmaceutical composition may include a blend of water and alcohol as the polar phase which has been thickened by suitable natural, modified natural, or synthetic polymers such as described below. Alternatively, the thickened hydroalcoholic gels can be thickened using suitable polyethoxylate alky chain surfactants or other nonionic, cationic, or anionic systems. The alcohol can be ethanol, isopropyl alcohol or other pharmaceutically acceptable alcohol. In embodiments of the present invention, the amount of alcohol (e.g., ethanol) can be less than 20% and still provide a self-preserving formulation.

Hydrophilic gels: The pharmaceutical composition may be a system in which the continuous phase includes at least one water soluble or water dispersible hydrophilic component other than water. The formulation may optionally also contain water up to 60% by weight. Higher levels may be suitable in some compositions. Suitable hydrophilic components include one or more glycols such as polyols such as glycerin, propylene glycol, butylene glycols, polyethylene glycols (PEG), random or block copolymers of ethylene oxide, propylene oxide, and/or butylene oxide, polyalkoxylated surfactants having one or more hydrophobic moieties per molecule, silicone copolyols, blend of ceteareth-6 and stearyl alcohol as well as combinations thereof, and the like.

In addition to the active ingredient, the formulation may contain additional excipients commonly present in such dosage forms. Such excipients will vary depending on the type of the dosage form and the desired characteristics. The pharmaceutical compositions for use with the methods disclosed herein may include one or more solvents, moisturizer, surfactant and emulsifier, polymer and thickener or additional excipients. In certain embodiments, the pharmaceutical composition comprises roflumilast and one or more of diethylene glycol monoethyl ether, an emulsifier blend of cetearyl alcohol, dicetyl phosphate, and ceteth-10 phosphate, and hexylene glycol.

### Solvents

In certain embodiments, the pharmaceutical composition may include one or more solvents or co-solvents to obtain the desired level of active ingredient solubility in the topical product. The solvent may also modify skin permeation or the activity of other excipients contained in the formulation. Solvents include but are not limited to acetone, ethanol, benzyl alcohol, butyl alcohol, diethyl sebacate, diethylene glycol monoethyl ether, diisopropyl adipate, dimethyl isosorbide, dimethyl sulfoxide, ethyl acetate, isopropyl alcohol, isopropyl isostearate, isopropyl myristate, N-methyl pyrrolidinone, polyethylene glycol, glycerol, propylene glycol and SD alcohol. In certain embodiments, the solvent is selected from the group consisting of 1,3-butylene glycol, 1,2-hexanediol, 1,3-propanediol, 1,2-pentanediol (also known as pentylene glycol), dipropylene glycol, 2-(2-butoxy-ethoxy) ethanol (also known as butoxydiglycol), 1,6-hexanediol, propylene glycol methyl ethyl acetate (also known as PGMEA or 1-methoxy-2-propanol acetate), 5-methyloxolan-2-one (also known as gamma-valerolactone), pantolactone, 1,3-butanediol, 1,5-pentanediol, 1,6-hexanediol, 1-heptanol, 1-hexanol, 2-(2-ethoxyethoxyl) ethyl acetate, 2-(2-methoxyethoxy) ethanol, 2-butoxyethanol, 2-butoxyethyl acetate, 2-ethoxyethanol, 2-ethoxyethyl acetate, 2-methoxyethanol, diethylene glycol dimethyl ether (also known as bis(2-methoxyethyl) ether), diethylene glycol, propylene glycol methyl ether, and combinations thereof. In certain embodiments, the solvent is selected from the group consisting of 1,3-butylene glycol, 1,2-hexanediol, 1,3-propanediol, 1,2-pentanediol, dipropylene glycol, 2-(2-butoxy-ethoxy) ethanol, 1,6-hexanediol, propylene glycol methyl ethyl acetate, 5-methyloxolan-2-one, and pantolactone. When treating a patient with an inflammatory condition, the solvent is preferably not ethanol, isopropyl alcohol or denatured alcohol.

### Moisturizers

In certain embodiments, the pharmaceutical composition may include a moisturizer to increase the level of hydration. The moisturizer can be a hydrophilic material including humectants or it can be a hydrophobic material including emollients. Suitable moisturizers include but are not limited to: 1,2,6-hexanetriol, 2-ethyl-1,6-hexanediol, butylene glycol, glycerin, polyethylene glycol 200-8000, butyl stearate, cetostearyl alcohol, cetyl alcohol, cetyl esters wax, cetyl palmitate, cocoa butter, coconut oil, cyclomethicone, dimethicone, docosanol, ethylhexyl hydroxystearate, fatty acids, glyceryl isostearate, glyceryl laurate, glyceryl monostearate, glyceryl oleate, glyceryl palmitate, glycol distearate, glycol stearate, isostearic acid, isostearyl alcohol, lanolin, mineral oil, limonene, medium-chain triglycerides, menthol, myristyl alcohol, octyldodecanol, oleic acid, oleyl alcohol, oleyl oleate, olive oil, paraffin, peanut oil, petrolatum, Plastibase-50W, white petrolatum, isopropyl palmitate, and stearyl alcohol.

### Surfactants and Emulsifiers

In certain embodiments, the pharmaceutical composition may include one or more surfactants to emulsify the composition and to help wet the surface of the actives or excipients. As used herein the term “surfactant” means an amphiphile (a molecule possessing both polar and nonpolar regions which are covalently bound) capable of reducing the surface tension of water and/or the interfacial tension between water and an immisicible liquid. Surfactants include but are not limited to alkyl aryl sodium sulfonate, Amerchol-CAB, ammonium lauryl sulfate, apricot kernel oil PEG-6 esters, Arlacel, benzalkonium chloride, Ceteareth-6, Ceteareth-12, Ceteareth-15, Ceteareth-30, cetearyl alcohol/ceteareth-20, cetearyl ethylhexanoate, ceteth-10, ceteth-2, ceteth-20, ceteth-23, choleth-24, cocamide ether sulfate, cocamine oxide, coco betaine, coco diethanolamide, coco monoethanolamide, coco-caprylate/caprate, disodium cocoamphodiacetate, disodium laureth sulfosuccinate, disodium lauryl sulfoacetate, disodium lauryl sulfosuccinate, disodium oleamido monoethanolamine sulfosuccinate, docusate sodium, laureth-2, laureth-23, laureth-4, lauric diethanolamide, lecithin, mehoxy PEG-16, methyl gluceth-10, methyl gluceth-20, methyl glucose sesquistearate, oleth-2, oleth-20, PEG 6-32 stearate, PEG-100 stearate, PEG-12 glyceryl laurate, PEG-120 methyl glucose dioleate, PEG-15 cocamine, PEG-150 distearate, PEG-2 stearate, PEG-20 methyl glucose sesqustearate, PEG-22 methyl ether, PEG-25 propylene glycol stearate, PEG-4 dilaurate, PEG-4 laurate, PEG-45/dodecyl glycol copolymer, PEG-5 oleate, PEG-50 Stearate, PEG-54 hydrogenated castor oil, PEG-6 isostearate, PEG-60 hydrogenated castor oil, PEG-7 methyl ether, PEG-75 lanolin, PEG-8 laurate, PEG-8 stearate, Pegoxol 7 stearate, pentaerythritol cocoate, poloxamer 124, poloxamer 181, poloxamer 182, poloxamer 188, poloxamer 237 poloxamer 407, polyglyceryl-3 oleate, polyoxyethylene alcohols, polyoxyethylene fatty acid esters, polyoxyl 20 cetostearyl ether, polyoxyl 40 hydrogenated castor oil, polyoxyl 40 stearate, polyoxyl 6 and polyoxyl 32, polyoxyl glyceryl stearate, polyoxyl stearate, polysorbate 20, polysorbate 40, polysorbate 60, polysorbate 65, polysorbate 80, PPG-26 oleate, PROMULGEN™ 12, propylene glycol diacetate, propylene glycol dicaprylate, propylene glycol monostearate, sodium xylene sulfonate, sorbitan monooleate, sorbitan monopalmitate, sorbitan monostearate, steareth-2, steareth-20, steareth-21, steareth-40, tallow glycerides, and emulsifying wax. Preferably, the emulsifier is a self-emulsifying wax blend of dicetyl phosphate and ceteth-10 phosphate.

### Polymers and Thickeners

In certain embodiments, the pharmaceutical composition may include a soluble, swellable, or insoluble organic polymeric thickeners such as natural and synthetic polymers or inorganic thickeners such as acrylates copolymer, carbomer 1382, carbomer copolymer type B, carbomer homopolymer type A, carbomer homopolymer type B, carbomer homopolymer type C, carboxy vinyl copolymer, carboxymethylcellulose, carboxypolymethylene, carrageenan, guar gum, hydroxyethyl cellulose, hydroxypropyl cellulose, microcrystalline wax, and methylcellulose,

### Additional Excipients

In certain embodiments, the pharmaceutical composition may include additional excipients such as fillers, carriers and excipients conventionally found in cosmetic and pharmaceutical topical products. In a preferred embodiment, the fillers, carriers and excipients are suitable for topical administration. Additional excipients including but not limited to antifoaming agents, preservatives (e.g. p-hydroxybenzoic esters, benzyl alcohol, phenylmercury salts, chlorocresol, methylparaben, propylparaben), antioxidants, sequestering agents, stabilizers, buffers, pH adjusting solutions, skin penetration enhancers, film formers, dyes, pigments, diluents, bulking agents, fragrances and other excipients to improve the stability or aesthetics, may be added to the composition.

Compositions according to the present invention may be formulated with additional active agents depending on other conditions being treated. The additional active agents include but are not limited to NSAIDs (e.g. Aspirin, Ibuprofen, Ketoprofen, Naproxen), Apremilast, JAK inhibitors (e.g. Tofacitinib, Ruxolitinib, Oclacit), leukotriene inhibitors (e.g. Zileuton, Zafirlukast, Montelukast), mast cell stabilizers (e.g. Nedocromil, Cromolyn sodium, Ketotifen, Pemirolast), Anthralin (dithranol), Azathioprine, Tacrolimus, Pimecrolimus, Coal tar, Methotrexate, Methoxsalen, Salicylic acid, Ammonium lactate, Urea, Hydroxyurea, 5-fluorouracil, Propylthouracil, 6-thioguanine, Sulfasalazine, Mycophenolate mofetil, Fumaric acid esters, Corticosteroids (e.g. Aclometasone, Amcinonide, Betamethasone, Clobetasol, Clocotolone, Mometasone, Triamcinolone, Fluocinolone, Fluocinonide, Flurandrenolide, Diflorasone, Desonide, Desoximetasone, Dexamethasone, Halcinonide, Halobetasol, Hydrocortisone, Methylprednisolone, Prednicarbate, Prednisone), Corticotropin, Vitamin D analogues (e.g. calcipotriene, calcitriol), Acitretin, Tazarotene, Cyclosporine, Resorcinol, Tapinarof, Colchicine, bronchodilators (e.g. beta-agonists, anticholinergics, theophylline), and antibiotics (e.g. erythromycin, ciprofloxacin, metronidazole). Alternatively, the additional active agent can be administered as a separate composition.

Compositions according to the present invention may be formulated with additional antifungal agents according to the specific fungal infection being treated. The additional antifungal agents include but are not limited to: drugs containing miconazole (Daktarin, Micatin & Monistat), ciclopirox olamine (Batrafen, Loprox, Penlac, and Stieprox), clotrimazole (Canesten, Hydrozole), butenafine (Lotrimin Ultra, Mentax), terbinafine (Lamisil, Terbisil, Zabel), amorolfine (Curanail, Loceryl, Locetar, and Odenil), naftifine (Naftin), tolnaftate (Tinactin), ketoconazole (Nizoral), griseofulvin, imidazoles (bifonazole, clomidazole, econazole, fenticonazole, isoconazole, miconazole, oxiconazole, sertaconazole, sulconazole, tioconazole), triazole (fluconazole, itraconazole, posaconazole (Noxafil), voriconazole (Vfend)), benzimidazole (thiabendazole), ethylparaben, flucytosine, salicylic acid, selenium sulfide, and undecylenic acid. Alternatively, the additional anti-fungal agent can be administered as a separate composition.

Compositions according to the present invention may be formulated with common topical anti-inflammatory agents including, but not limited to, Diflucortolone valerate, Fluocinonide, Flurandrenolide, Halobetasol propionate, Amcinonide, Desoximetasone, Diflorasone, Halcinonide, Betamethasone valerate, Diflorasone diacetate, Fluticasone propionate, Mometasone furoate, Triamcinolone acetonide, Clocortolone pivalate, Fluocinolone acetonide, Fluticasone propionate, Hydrocortisone valerate, Mometasone furoate, Desonide, Hydrocortisone butyrate, Hydrocortisone probutate, Hydrocortisone valerate, Prednicarbate, Betamethasone dipropionate augmented Clobetasol propionate, Alclometasone dipropionate, Hydrocortisone (base, ≥2%), Hydrocortisone (base, <2%), calcineurin inhibitors and Hydrocortisone acetate. Alternatively, the anti-inflammatory agent can be administered as a separate composition.

### Methods of Manufacture

The topical pharmaceutical compositions may be prepared by processes typically used in the field of manufacture of pharmaceutical formulations for topical application. In order to make a single-phase formulation, such as a liquid, the constituents of the formulation may be combined and mixed until a homogenous solution or suspension of the active ingredient is obtained. In order to make a multiphase formulation such as an emulsion, for example, the components of the aqueous phase and of the oil phase may be separately combined and mixed until homogenous solutions are obtained and then the aqueous solution and the oil solution may be combined and mixed, such as by shear mixing, to form the formulation. The one or more drug actives may be dissolved (molecularly dispersed), complexed, or associated with an excipient or other active, or may be particulate (amorphous or crystalline). The oil phase may be added to the water phase, or the water phase may be added to the oil phase. The phases may be combined and mixed, such as at elevated temperatures of 50-90° C. or at room temperature, that is between 20-30° C., or at a temperature between room temperature and the elevated temperatures.

The following examples illustrate certain embodiments of the invention without limitation.

### EXAMPLES

While various embodiments have been described above, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

### Example 1

A roflumilast cream having Formulation 1 as disclosed in Table 2 was prepared.

TABLE 2
Composition of Formulation 1

| | Concentration |
|---|---|
| Ingredient | (% w/w) |
| Roflumilast | 0.3% |
| White Petrolatum | 10.0% |
| Isopropyl Palmitate | 5.0% |
| Crodafos CES | 10% |
| (blend of cetearyl alcohol, dicetyl phosphate, | |
| ceteth-10 phosphate) | |
| Hexylene glycol | 2.0% |
| Transcutol P | 25% |
| (diethylene glycol monoethyl ether) | |
| Methylparaben | 0.2% |
| Propylparaben | 0.05% |
| Purified water | q.s. ad 100 |

### Example 2

A roflumilast cream having Formulation 2 as disclosed in Table 3 was prepared.

TABLE 3
Composition of Formulation 2

| | Concentration |
|---|---|
| Ingredient | (% w/w) |
| Roflumilast | 0.15% |
| White Petrolatum | 10.0% |
| Isopropyl Palmitate | 5.0% |
| Crodafos CES | 10% |
| (blend of cetearyl alcohol, dicetyl phosphate, | |
| ceteth-10 phosphate) | |
| Hexylene glycol | 2.0% |
| Transcutol P | 25% |
| (diethylene glycol monoethyl ether) | |
| Methylparaben | 0.2% |
| Propylparaben | 0.05% |
| Purified water | q.s. ad 100 |

### Example 3

A roflumilast cream having Formulation 3 as disclosed in Table 4 was prepared.

TABLE 4
Composition of Formulation 3

| | Concentration |
|---|---|
| Ingredient | (% w/w) |
| Roflumilast | 0.05% |
| White Petrolatum | 10.0% |
| Isopropyl Palmitate | 5.0% |
| Crodafos CES | 10% |
| (blend of cetearyl alcohol, dicetyl phosphate, | |
| ceteth-10 phosphate) | |
| Hexylene glycol | 2.0% |
| Transcutol P | 25% |
| (diethylene glycol monoethyl ether) | |
| Methylparaben | 0.2% |
| Propylparaben | 0.05% |
| Purified water | q.s. ad 100 |

### Example 4

A roflumilast foam having Formulation 4 as disclosed in Table 5 was prepared.

TABLE 5
Composition of Formulation 4

| | Concentration |
|---|---|
| Ingredient | (% w/w) |
| Roflumilast | 0.3% |
| White Petrolatum | 5.0% |
| Isopropyl Palmitate | 2.5% |
| Crodafos CES | 2.0% |
| (blend of cetearyl alcohol, dicetyl phosphate, | |
| ceteth-10 phosphate) | |
| Hexylene glycol | 2.0% |
| Transcutol P | 25% |
| (diethylene glycol monoethyl ether) | |
| Methylparaben | 0.2% |
| Propylparaben | 0.05% |
| Purified water | q.s. ad 100 |
| Hydrocarbon aerosol propellant | 8-grams of |
| | propellant per |
| | cannister |

### Example 5

A roflumilast cream having Formulation 5 as disclosed in Table 6 will be prepared.

TABLE 6
Composition of Formulation 5

| | Concentration |
|---|---|
| Ingredient | (% w/w) |
| Roflumilast | 0.3% |
| White Petrolatum | 10.0% |
| Isopropyl Palmitate | 5.0% |
| Crodafos CES | 10% |
| (blend of cetearyl alcohol, dicetyl phosphate, | |
| ceteth-10 phosphate) | |
| Hexylene glycol | 2.0% |
| Transcutol P | 25% |
| (diethylene glycol monoethyl ether) | |
| Purified water | q.s. ad 100 |

### Example 6

Two Phase 3, parallel group, double blind, vehicle-controlled studies in which a topical roflumilast cream 0.15% or vehicle was applied QD for 4 weeks to subjects 6 years of age and older with mild to moderate atopic dermatitis were conducted. At entry, subjects had ≥3% BSA involvement (excluding the scalp, palms, soles) and mild or moderate atopic dermatitis (AD) based on vIGA-AD assessment. Upon determination of eligibility, subjects were randomized 2:1 to either ARQ-151 cream 0.15% or matching vehicle cream. The randomization was stratified by vIGA-AD score at Baseline/Day 1 (‘Mild’ vs. ‘Moderate’) and by study site. Subjects/caregivers applied ARQ-151 cream 0.15% or vehicle cream QD to all AD affected areas and any newly appearing AD lesions that arose during the study, except on the scalp. Subjects/caregivers maintained treatment of these areas with study drug for the duration of the study regardless of whether treatable areas of AD cleared prior to Week 4/Day 29. At the Week 4 visit, subjects were eligible to enroll in a 12-month, open label extension study evaluating topical roflumilast cream 0.15% QD.

The study objective was to assess the safety and efficacy of the topical roflumilast cream 0.15% versus vehicle administered QD for 4 weeks to individuals with atopic dermatitis. Subjects were male and female children and adolescents (6-17 years), and adults (≥18 years). Subjects had mild to moderate atopic dermatitis involvement with a vIGA-AD score of ‘2’ (Mild) or ‘3’ (Moderate) for study entry. Up to 50% of the subjects were ≥18 years old. Approximately 650 subjects were randomized in this study.

The primary efficacy endpoint was IGA Success, defined as a vIGA-AD score of ‘clear’ or ‘almost clear’ PLUS a 2-grade improvement from Baseline at Week 4.

The secondary efficacy endpoints were: (1) in subjects with a vIGA-AD score of ‘Moderate’ at randomization, vIGA-AD Success at Week 4; (2) in subjects with Baseline WI-NRS>4, achievement of at least a 4-point reduction on the WI-NRS at Week 4; (3) in subjects with Baseline WI-NRS≥4, achievement of at least a 4-point reduction on the WI-NRS at Week 2; (4) in subjects with baseline WI-NRS≥4, achievement of at least a 4-point reduction on the WI-NRS at Week 1; (5) achievement of at least a 75% reduction in the Eczema Area and Severity Index (EASI-75) at Week 4; (6) vIGA-AD of ‘clear’ or ‘almost clear’ at Week 4; (7) vIGA-AD Success at Week 2; (8) vIGA-AD Success at Week 1; (9) vIGA-AD of ‘clear’ or ‘almost clear’ at Week 2; and (10) vIGA-AD of ‘clear’ or ‘almost clear’ at Week 1.

The results of the studies are set forth in Tables 7 and 8. The results in these tables report results from Day 2, wherein Day 1 is the studies' baseline visit. Thus, Day 2 is the day following baseline and represents patient results 1 day after the start of the study.

TABLE 7
Change from Baseline and Percent Change from Baseline
in Daily WI-NRS Observed Data at Study Day 2 (Study 1)

| | | |
|---|---|---|
| | Roflumilast Cream 0.15% | Vehicle |
| Statistic | (N = 433) | (N = 221) |

Value

| N | 419 | | 213 | |
|---|---|---|---|---|
| Mean (SD) | 5.1 | (2.52) | 5.4 | (2.73) |

| | | |
|---|---|---|
| Median | 5.0 | 6.0 |
| Q1, Q3 | 3.0, 7.0 | 3.0, 8.0 |
| Min, Max | 0, 10 | 0, 10 |

Change from Baseline

| N | 417 | | 212 | |
|---|---|---|---|---|
| Mean (SD) | −0.6 | (1.88) | −0.2 | (1.80) |
| Median | 0.0 | | 0.0 | |
| Q1, Q3 | −1.0, 0.0 | | −1.0, 1.0 | |
| Min, Max | −7, 7 | | −8, 6 | |
| LS mean (SE) | −0.6 | (0.10) | −0.2 | (0.13) |
| LS mean difference (SE) | −0.4 | (0.15) | | |

| | |
|---|---|
| 95% CI for mean | (−0.70, −0.13) |
| difference | |
| p-value | 0.0042 |

Percent Change from Baseline

| N | 402 | | 203 | |
|---|---|---|---|---|
| Mean (SD) | −5.90 | (44.285) | 3.87 | (60.967) |
| Median | −10.0 | | 0.00 | |
| Q1, Q3 | −28.57, 0.00 | | −16.67, 14.29 | |
| Min, Max | −100.0, 350.0 | | −100.0, 600.0 | |
| LS mean (SE) | −5.83 | (2.814) | 2.90 | (3.681) |
| LS mean difference (SE) | −8.73 | (4.188) | | |

| | |
|---|---|
| 95% CI for mean | (−16.956, −0.502) |
| difference | |
| p-value | 0.0376 |

TABLE 8
Change from Baseline and Percent Change from Baseline
in Daily WI-NRS Observed Data at Study Day 2 (Study 2)

| | | |
|---|---|---|
| | Roflumilast Cream 0.15% | Vehicle |
| Statistic | (N = 451) | (N = 232) |

Value

| N | 440 | | 225 | |
|---|---|---|---|---|
| Mean (SD) | 5.2 | (2.60) | 5.6 | (2.46) |

| | | |
|---|---|---|
| Median | 5.0 | 6.0 |
| Q1, Q3 | 3.0, 7.0 | 4.0, 7.0 |
| Min, Max | 0, 10 | 0, 10 |

Change from Baseline

| N | 439 | | 225 | |
|---|---|---|---|---|
| Mean (SD) | −0.9 | (2.08) | −0.2 | (1.91) |
| Median | −1.0 | | 0.0 | |
| Q1, Q3 | −2.0, 0.0 | | −1.0, 1.0 | |
| Min, Max | −10, 5 | | −7, 5 | |
| LS mean (SE) | −0.8 | (0.10) | −0.2 | (0.14) |
| LS mean difference (SE) | −0.6 | (0.15) | | |

| | |
|---|---|
| 95% CI for mean | (−0.86, −0.26) |
| difference | |
| p-value | 0.0003 |

Percent Change from Baseline

| N | 429 | | 220 | |
|---|---|---|---|---|
| Mean (SD) | −7.64 | (54.120) | 3.75 | (45.893) |
| Median | −12.50 | | 0.00 | |
| Q1, Q3 | −25.00, 0.00 | | −20.00, 15.48 | |
| Min, Max | −100.0, 500.0 | | −87.5, 250.0 | |
| LS mean (SE) | −6.76 | (2.701) | 2.07 | (3.597) |
| LS mean difference (SE) | −8.83 | (4.061) | | |

| | |
|---|---|
| 95% CI for mean | (−16.808, −0.856) |
| difference | |
| p-value | 0.0300 |

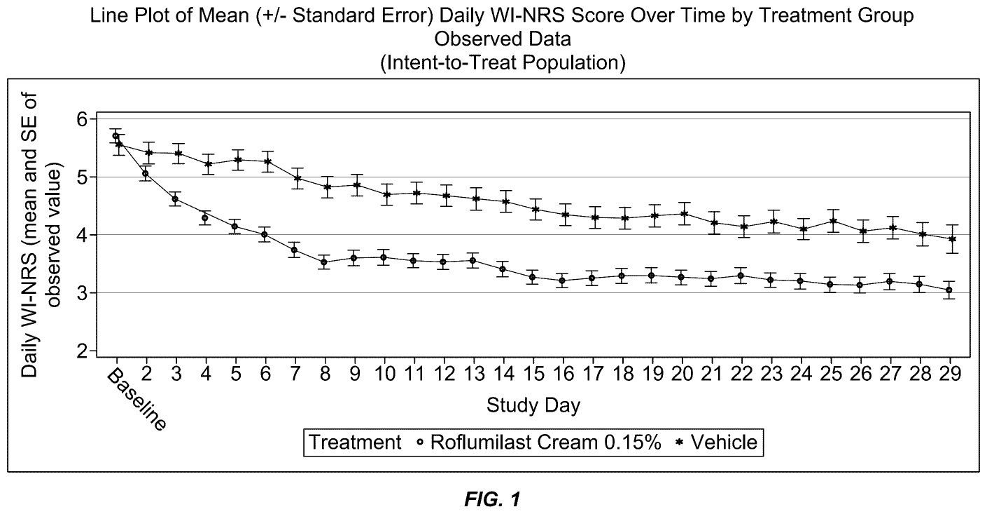

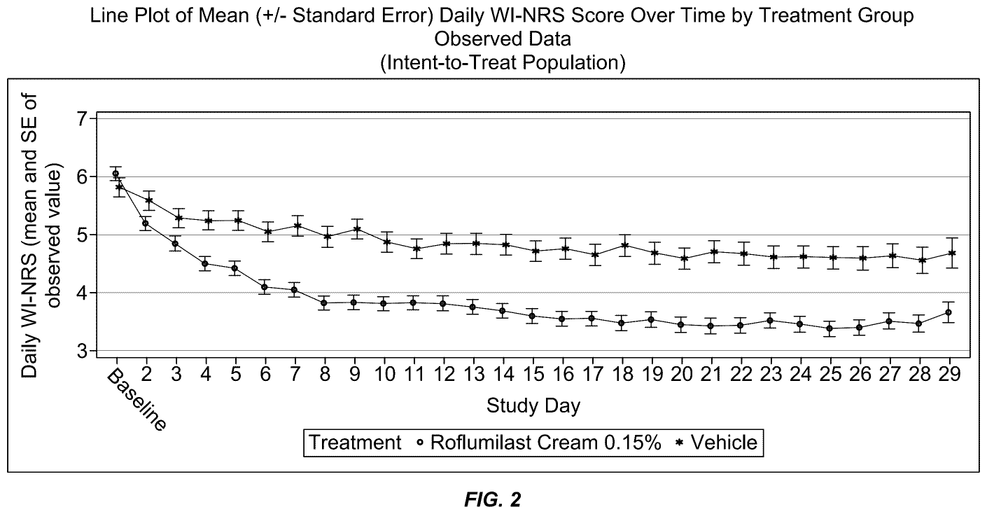

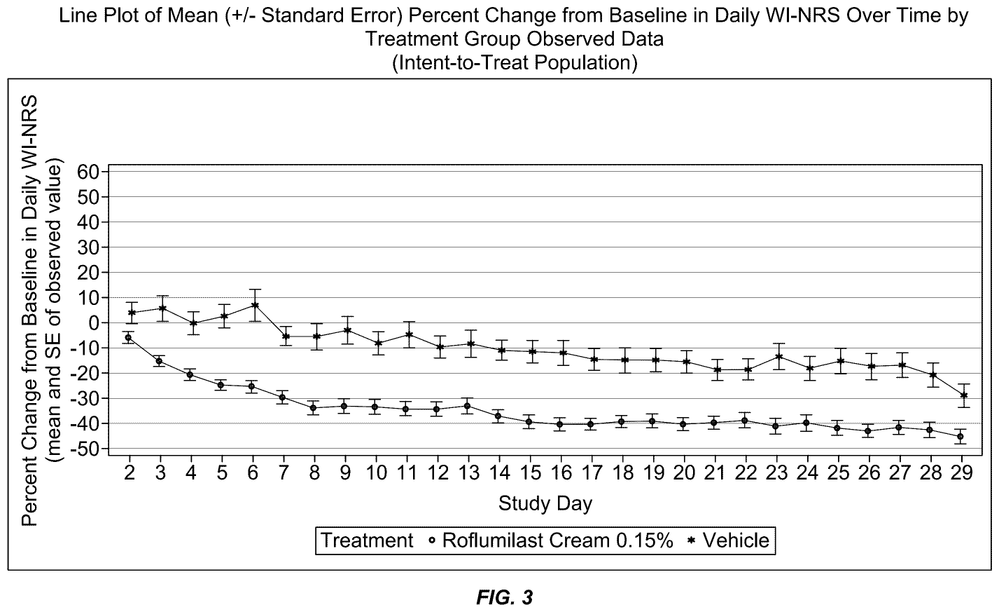

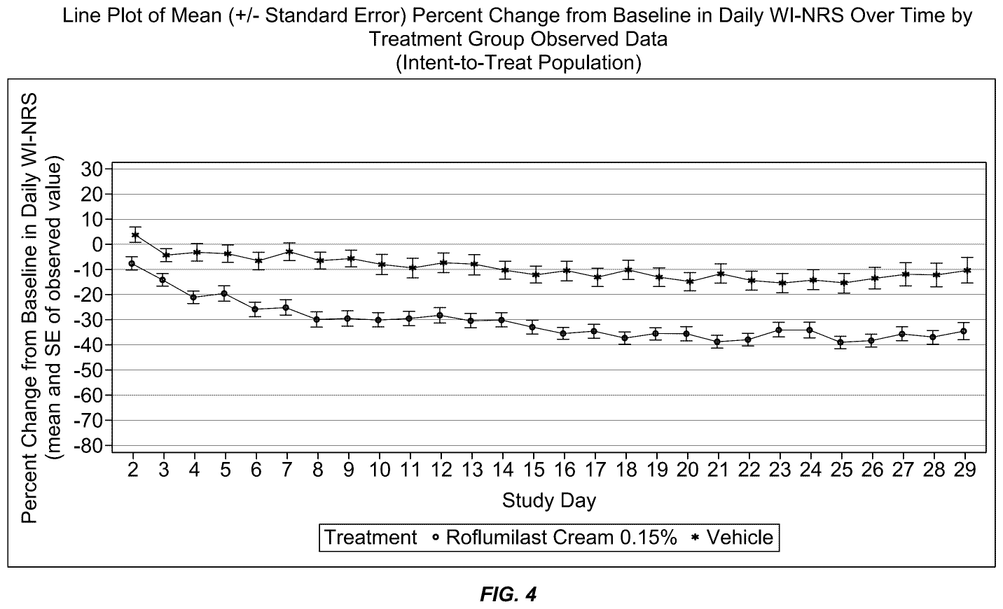

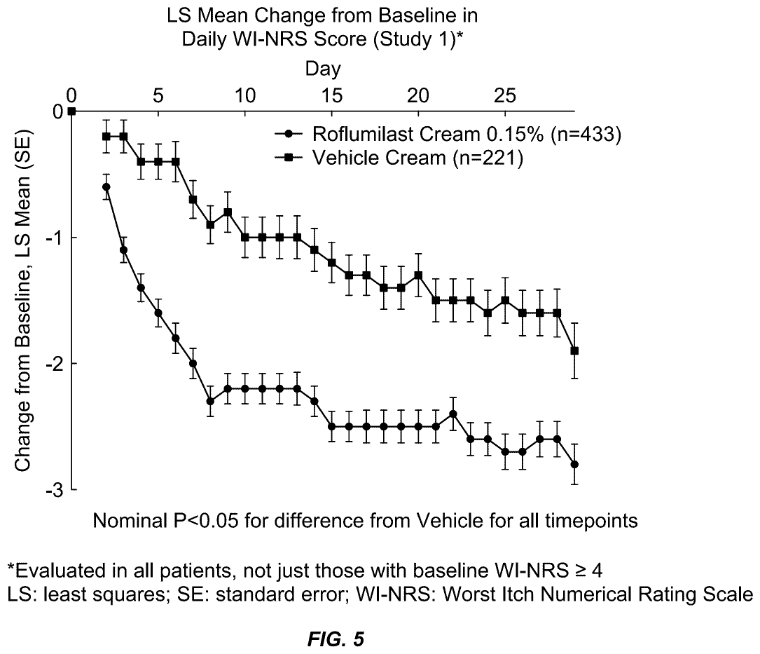

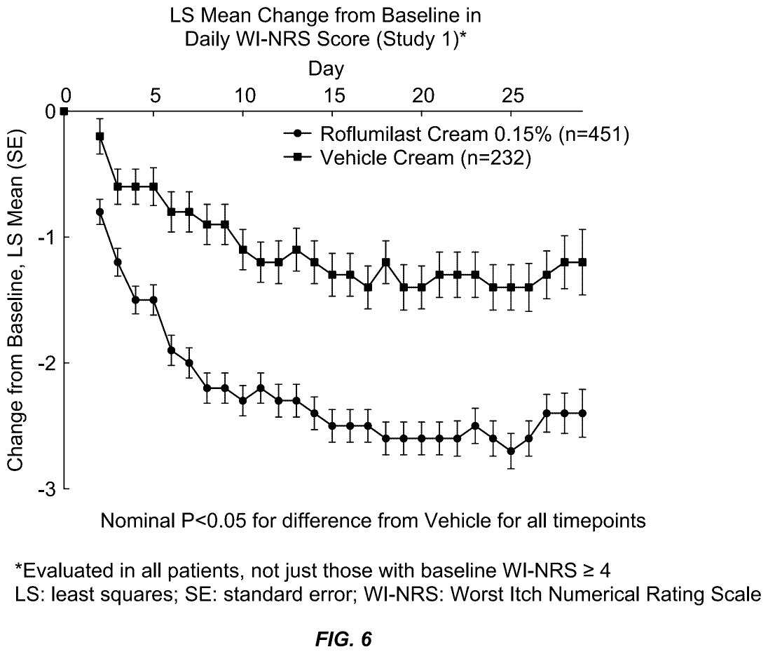

The results of the studies are also set forth in FIGS. 1-6. FIG. 1 provides a line plot of the mean daily WI-NRS score over time by treatment group (roflumilast cream 0.15% versus vehicle) from Study 1. FIG. 2 provides a line plot of the mean daily WI-NRS score over time by treatment group (roflumilast cream 0.15% versus vehicle) from Study 2. FIG. 3 provides a line plot of the percent change from baseline in daily WI-NRS over time for two treatment groups (roflumilast cream 0.15% versus vehicle) from Study 1. FIG. 4 provides a line plot of the percent change from baseline in daily WI-NRS over time for two treatment groups (roflumilast cream 0.15% versus vehicle) from Study 2. FIG. 5 provides a line plot of the mean change from baseline in WI-NRS score from Study 1. FIG. 6 provides a line plot of the mean change from baseline in WI-NRS score from Study 2. As illustrated in FIGS. 5 and 6, an improvement in itch with roflumilast cream 0.15% was observed at 1 day after first application (p<0.05).

Over 30% of individuals treated with roflumilast cream in each study achieved WI-NRS Success defined as at least a 4-point reduction in WI-NRS from baseline by Week 4. In addition, a daily improvement in itch was observed in those treated with roflumilast cream with a significant improvement at 1 day following the first application (p<0.05) as measured by WI-NRS.

Both studies met the primary endpoint of IGA Success, defined as a validated Investigator Global Assessment—Atopic Dermatitis (vIGA-AD) score of ‘clear’ or ‘almost clear’ plus a 2-grade improvement from baseline at Week 4 (Study 1:32.0% roflumilast cream vs. 15.2% vehicle, P<0.0001; Study 2:28.9% roflumilast cream vs. 12.0% vehicle, P<0.0001). In addition, rapid and significant improvements in v-IGA success were demonstrated as early as Week 2 (Study 1:21.2% for roflumilast cream vs. 6.4% for vehicle; P<0.0001; Study 2:17.7% for roflumilast cream vs 5.3% for vehicle; P<0.0001).

Roflumilast cream also demonstrated rapid and statistically significant improvements compared to vehicle on key secondary endpoints, with more than 40% of children age 6 years and older and adults treated with roflumilast cream achieving a 75% reduction in Eczema Area and Severity Index (EASI-75) at Week 4 compared to vehicle (Study 1:43.2% vs. 22.0%, P<0.0001; Study 2:42.0% vs. 19.7%, P<0.0001). Additionally, significant improvements in EASI-75 were observed with roflumilast cream as early as Week 1 in both studies compared to vehicle (Study 1:14.0% vs. 5.5%, p=0.0006; Study 2:13.3% vs. 7.8%, p=0.0329). In both studies, approximately 40% of children and adults treated with roflumilast cream achieved a vIGA-AD score of Clear (0) or Almost Clear (1) at Week 4 (Study 1:41.5% vs. 25.2%, P<0.0001; Study 2: 39% vs. 16.9%, P<0.0001).

### Example 7

A Phase 3 vehicle-controlled study in which a topical roflumilast cream 0.05% or vehicle was applied QD for 4 weeks to subjects 2 to 5 years of age with mild to moderate atopic dermatitis was conducted. The study objective was to assess the safety and efficacy of the topical roflumilast cream 0.05% versus vehicle administered QD for 4 weeks to children 2 to 5 years of age with atopic dermatitis. Subjects were male and female children 2 to 5 years of age. Subjects had mild to moderate atopic dermatitis involvement with a vIGA-AD score of ‘2’ (Mild) or ‘3’ (Moderate) for study entry. Subjects had a mean Body Surface Area (BSA) of 22% overall, and a range of 3% to 82%. Approximately 650 subjects were randomized in this study.

The results of the studies are set forth in Table 9. The results in this table report results from Day 2, wherein Day 1 is the studies' baseline visit. Thus, Day 2 is the day following baseline and represents patient results 1 day after the start of the study.

TABLE 9
Change from Baseline in Daily WI-
NRS Observed Data at Study Day 2

| | | |
|---|---|---|
| | Roflumilast Cream 0.05% | Vehicle |
| Statistic | (N = 436) | (N = 215) |

Value

| N | 428 | | 209 | |
|---|---|---|---|---|
| Mean (SD) | 5.4 | (2.44) | 5.7 | (2.43) |

| | | |
|---|---|---|
| Median | 6.0 | 6.0 |
| Q1, Q3 | 4.0, 7.0 | 3.0, 8.0 |
| Min, Max | 0, 10 | 0, 10 |

Change from Baseline

| N | 426 | | 209 | |
|---|---|---|---|---|
| Mean (SD) | −0.8 | (1.93) | −0.2 | (1.58) |
| Median | 0.0 | | 0.0 | |
| Q1, Q3 | −1.0, 0.0 | | −1.0, 1.0 | |
| Min, Max | −7, 8 | | −5, 5 | |
| LS mean (SE) | −0.8 | (0.09) | −0.4 | (0.13) |
| LS mean difference (SE) | −0.4 | (0.14) | | |
| 97.5% CI for LS mean | (−0.76, −0.13) | | | |
| difference | | | | |

| | |
|---|---|
| 95% CI for mean | (−0.72, −0.17) |
| difference | |
| p-value | 0.0014 |

Percent Change from Baseline

| N | 413 | | 207 | |
|---|---|---|---|---|
| Mean (SD) | −6.49 | (56.626) | 3.46 | (41.098) |
| Median | −10.00 | | 0.00 | |
| Q1, Q3 | −25.00, 0.00 | | −16.67, 14.29 | |
| Min, Max | −100.0, 800.0 | | −71.4, 200.0 | |
| LS mean (SE) | −7.81 | (2.746) | −1.31 | (3.742) |
| LS mean difference (SE) | −6.50 | (4.195) | | |

| | |
|---|---|
| 95% CI for mean | (−14.743, 1.734) |
| difference | |
| p-value | 0.1216 |

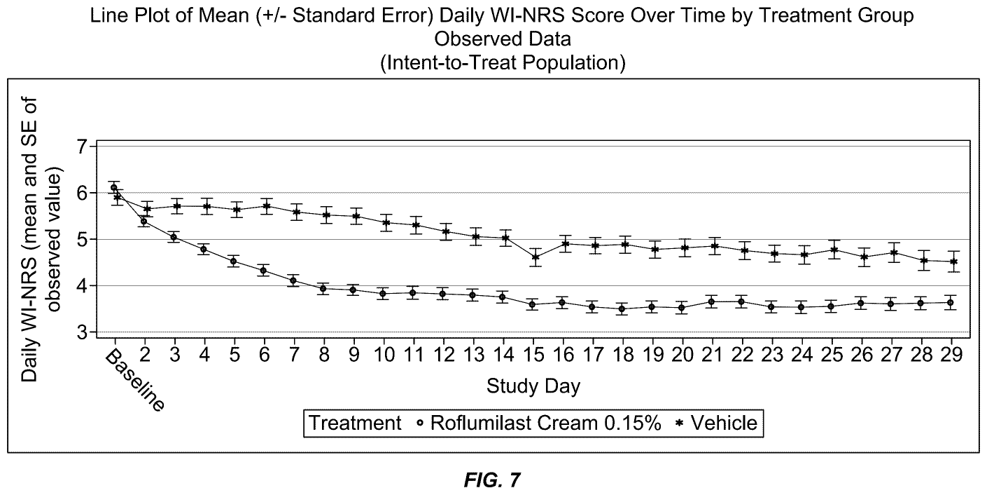

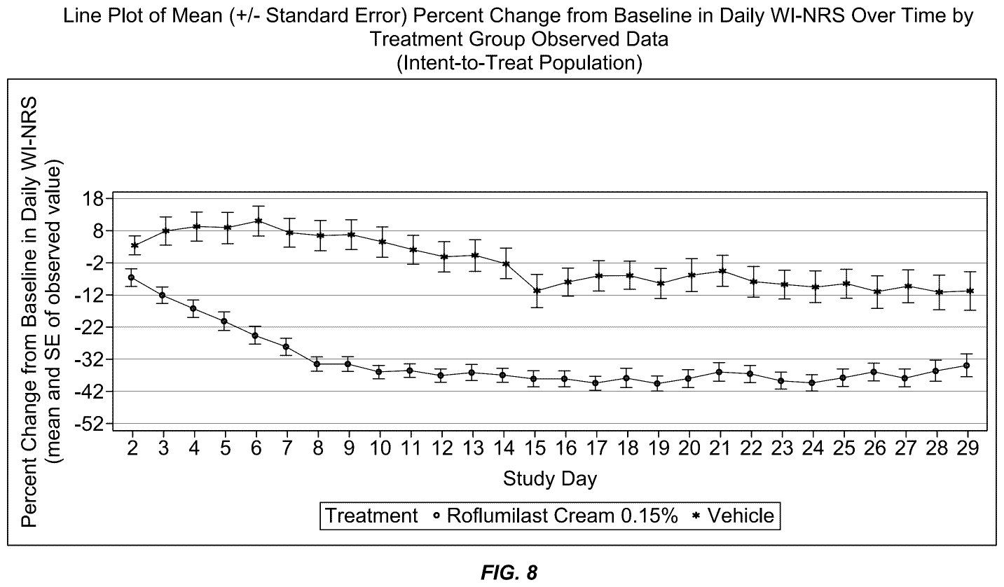

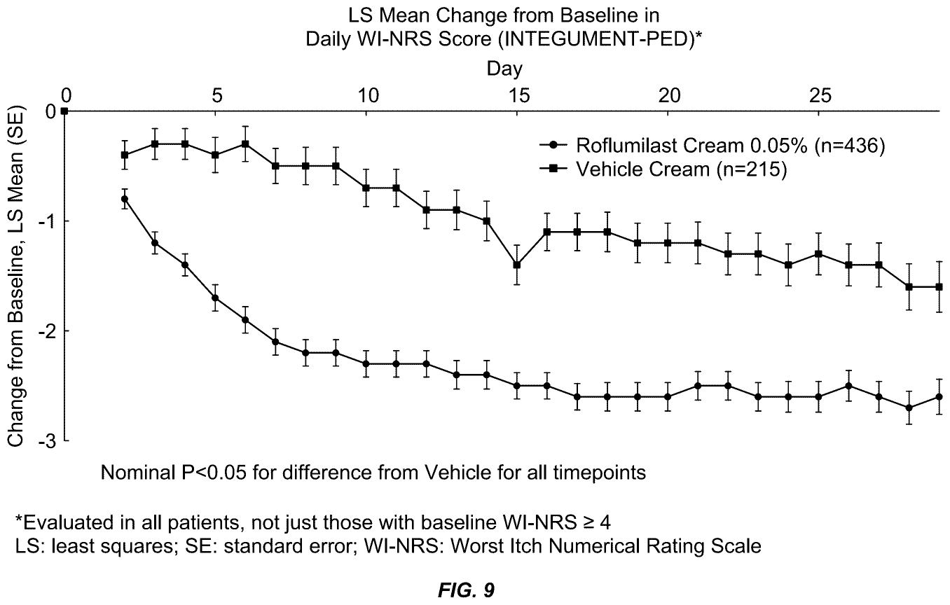

The results of the studies are also set forth in FIGS. 7, 8, and 9. FIG. 7 provides a line plot of the mean daily WI-NRS score over time by treatment group (roflumilast cream 0.05% versus vehicle). FIG. 8 provides a line plot of the percent change from baseline in daily WI-NRS over time for two treatment groups (roflumilast cream 0.05% versus vehicle). FIG. 9 provides a line plot of mean change from baseline in WI-NRS score over time by treatment group (roflumilast cream 0.05% versus vehicle). As illustrated in FIG. 9, an improvement in itch with roflumilast cream 0.05% was observed at 1 day after first application (p=0.0014).

Approximately 35.3% of children treated with roflumilast cream in the study achieved WI-NRS Success defined as at least a 4-point reduction in WI-NRS from baseline by Week 4. In addition, a daily improvement in itch was observed in those treated with roflumilast cream with a significant improvement at 1 day following the first application as measured by WI-NRS.

The study met the primary endpoint of IGA Success, defined as a validated Investigator Global Assessment-Atopic Dermatitis (vIGA-AD) score of ‘clear’ or ‘almost clear’ plus a 2-grade improvement from baseline at Week 4. For the primary endpoint, 25.4% of children treated once daily with roflumilast cream 0.05% achieved Investigator Global Assessment (IGA) Success compared to 10.7% of children treated with vehicle (P<0.0001) at Week 4, with significant improvements seen as early as Week 1. In the study, 39.4% of children treated with roflumilast cream 0.05% achieved a 75% improvement in EASI-75 at Week 4 compared to 20.6% treated with vehicle (P<0.0001). In addition, 35.3% of children treated with roflumilast cream achieved a four-point reduction in Worst Itch Numeric Scale (WI-NRS) at Week 4 (vs. 18.0% for vehicle-treated subjects [nominal P=0.0002]).

Roflumilast cream was very well-tolerated. Overall, the incidence of adverse events in the study was low, and the only adverse event occurring in ≥3% of subjects in either arm was upper respiratory tract infection. The most frequent adverse events in the roflumilast arm (≥2%) included upper respiratory tract infection, pyrexia, diarrhea, and vomiting. Of children who were randomized to roflumilast cream in the study, 93.8% completed the full four weeks, and there were few discontinuations due to adverse events (1.1% and 1.9% in the roflumilast cream and vehicle groups, respectively). The results reflect the favorable efficacy, safety, and tolerability of roflumilast cream for pediatric patients with atopic dermatitis.

### Example 8

A Phase 3 vehicle-controlled study in which a topical roflumilast foam 0.3% or vehicle was applied QD for 8 weeks to children aged 9 years and above and adults with moderate to severe seborrheic dermatitis was conducted. The study objective was to assess the safety and efficacy of the topical roflumilast foam 0.3% versus vehicle administered QD for 8 weeks in subjects with moderate to severe seborrheic dermatitis.

In the study, the WI-NRS was measured at baseline in patients and one and two days following administration of the roflumilast foam or vehicle. The results of the studies are set forth in Table 10 and Table 11. The results in Table 10 report results from Day 2 and the results in Table 11 report results from Day 3, wherein Day 1 is the studies' baseline visit. Thus, Day 2 is the day following baseline and represents patient results 1 day after the start of the study, and Day 3 is two days following baseline and represents patient results 2 days after the start of the study.

TABLE 10
Change from Baseline in Daily WI-
NRS Observed Data at Study Day 2

| | | |
|---|---|---|
| | Roflumilast | |
| | Foam 0.3% | Vehicle |
| Statistic | (N = 304) | (N = 153) |

Change from Baseline

| | | |
|---|---|---|
| LS Mean for Change from | −0.84 (0.186) | −0.64 (0.203) |
| Baseline (SE) | | |
| 99% Confidence Interval for LS | (−1.32, −0.35) | (−1.16, −0.11) |
| Mean Change from Baseline | | |
| 95% Confidence Interval for LS | (−1.20, −0.47) | (−1.03, −0.24) |
| Mean Change from Baseline | | |
| LS Mean in Difference from | −0.20 (0.163) | |
| Vehicle (SE) | | |
| 99% Confidence Interval for LS | (−0.62, 0.22) | |
| Mean Change from Vehicle | | |
| 95% Confidence Interval for LS | (−0.52, 0.12) | |
| Mean Change from Vehicle | | |
| p-value for Difference from | 0.2200 | |
| Vehicle | | |

Percent Change from Baseline

| | | |
|---|---|---|
| LS Mean for Change from | −18.20 (5.033) | −12.82 (5.421) |
| Baseline (SE) | | |
| 99% Confidence Interval for LS | (−31.23, −5.17) | (−26.86, 1.21) |
| Mean Change from Baseline | | |
| 95% Confidence Interval for LS | (−28.10, −8.30) | (−23.48, −2.16) |
| Mean Change from Baseline | | |
| LS Mean in Difference from | −5.38 (4.293) | |
| Vehicle (SE) | | |
| 99% Confidence Interval for LS | (−16.49, 5.74) | |
| Mean Change from Vehicle | | |
| 95% Confidence Interval for LS | (−13.82, 3.06) | |
| Mean Change from Vehicle | | |
| p-value for Difference from | 0.2111 | |
| Vehicle | | |

TABLE 11
Change from Baseline in Daily WI-
NRS Observed Data at Study Day 3

| | | |
|---|---|---|
| | Roflumilast | |
| | Foam 0.3% | Vehicle |
| Statistic | (N = 304) | (N = 153) |

Change from Baseline

| | | |
|---|---|---|
| LS Mean for Change from | −1.34 (0.200) | −0.90 (0.217) |
| Baseline (SE) | | |
| 99% Confidence Interval for LS | (−1.86, −0.82) | (−1.47, −0.34) |
| Mean Change from Baseline | | |
| 95% Confidence Interval for LS | (−1.73, −0.95) | (−1.33, −0.48) |
| Mean Change from Baseline | | |
| LS Mean in Difference from | −0.44 (0.175) | |
| Vehicle (SE) | | |
| 99% Confidence Interval for LS | (−0.89, 0.02) | |
| Mean Change from Vehicle | | |
| 95% Confidence Interval for LS | (−0.78, −0.09) | |
| Mean Change from Vehicle | | |
| p-value for Difference from | 0.0134 | |
| Vehicle | | |

Percent Change from Baseline

| | | |
|---|---|---|
| LS Mean for Change from | −27.87 (5.671) | −13.11 (6.111) |
| Baseline (SE) | | |
| 99% Confidence Interval for LS | (−42.55, −13.19) | (−28.93, 2.71) |
| Mean Change from Baseline | | |
| 95% Confidence Interval for LS | (−39.02, −16.72) | (−25.13, −1.09) |
| Mean Change from Baseline | | |
| LS Mean in Difference from | −14.76 (4.834) | |
| Vehicle (SE) | | |
| 99% Confidence Interval for LS | (−27.28, −2.25) | |
| Mean Change from Vehicle | | |
| 95% Confidence Interval for LS | (−24.27, −5.26) | |
| Mean Change from Vehicle | | |
| p-value for Difference from | 0.0024 | |
| Vehicle | | |

An improvement in itch was observed in those treated with roflumilast foam with patients experiencing a reduction in itch relative to vehicle at 1 and 2 days following the first application as measured by WI-NRS. The improvement in itch observed in those treated with roflumilast foam was significantly improved at 2 days following the first application (p<0.05) as measured by WI-NRS.

### Example 9

A Phase 3 vehicle-controlled study in which a topical roflumilast foam 0.3% or vehicle was applied QD for 8 weeks to subjects with scalp and body psoriasis ages 12 and older was conducted. A total of 432 subjects were enrolled in the study. The study objective was to assess the safety and efficacy of the topical roflumilast foam 0.3% versus vehicle administered QD for 8 weeks in subjects with scalp and body psoriasis.

In the study, the SI-NRS was measured at baseline in patients and one day later following administration of the roflumilast foam or vehicle. The results of the studies are set forth in Table 12. The results in this table report results from Day 2, wherein Day 1 is the studies' baseline visit. Thus, Day 2 is the day following baseline and represents patient results 1 day after the start of the study.

TABLE 12
Change from Baseline in Daily SI-
NRS Observed Data at Study Day 2

| | | |
|---|---|---|
| | Roflumilast | |
| | Foam 0.3% | Vehicle |
| Statistic | (N = 281) | (N = 151) |

Change from Baseline

| | | |
|---|---|---|
| LS Mean for Change from | −0.44 (0.127) | −0.01 (0.161) |
| Baseline (SE) | | |
| 97.5% Confidence Interval for | (−0.71, −0.14) | (−0.38, 0.35) |
| LS Mean Change from Baseline | | |
| 95% Confidence Interval for LS | (−0.67, −0.17) | (−0.33, 0.30) |
| Mean Change from Baseline | | |
| LS Mean in Difference from | −0.41 (0.157) | |
| Vehicle (SE) | | |
| 97.5% Confidence Interval for | (−0.76, −0.06) | |
| LS Mean Change from Vehicle | | |
| 95% Confidence Interval for LS | (−0.72, −0.10) | |
| Mean Change from Vehicle | | |
| p-value for Difference from | 0.0094 | |
| Vehicle | | |

Percent Change from Baseline

| | | |
|---|---|---|
| LS Mean for Change from | −4.92 (3.907) | 3.00 (5.014) |
| Baseline (SE) | | |
| 97.5% Confidence Interval for | (−13.71, 3.87) | (−8.29, 14.29) |
| LS Mean Change from Baseline | | |
| 95% Confidence Interval for LS | (−12.60, 2.76) | (−6.86, 12.86) |
| Mean Change from Baseline | | |
| LS Mean in Difference from | −7.92 (4.908) | |
| Vehicle (SE) | | |
| 97.5% Confidence Interval for | (−18.97, 3.13) | |
| LS Mean Change from Vehicle | | |
| 95% Confidence Interval for LS | (−17.58, 1.73) | |
| Mean Change from Vehicle | | |
| p-value for Difference from | 0.1075 | |
| Vehicle | | |

An improvement in scalp itch was observed in those treated with roflumilast foam with patients experiencing a reduction in itch relative to vehicle at 1 day following the first application as measured by SI-NRS.

### Example 10

A 52-week, phase 3, multicenter, open-label extension (“OLE”) of the trial described in Example 6 was conducted in adults and children ≥2 years of age with AD. Patients who had received vehicle control in the Example 6 were switched to receiving roflumilast cream in this extension. The study objective was to assess the long-term safety in a multicenter, open-label, single-arm study in subjects with atopic dermatitis with roflumilast cream 0.15% QD (subjects greater than or equal to 6 years of age) or roflumilast cream 0.05% QD (subjects 2 to 5 years of age) after completion of a prior Phase 3 study. The efficacy endpoints included Validated Investigator Global Assessment Atopic Dermatitis (vIGA-AD) value 0 or 1 at each assessment, and vIGA Success, defined as a vIGA-AD score of ‘clear’ or ‘almost clear’ PLUS a 2-grade improvement from Baseline. vIGA-AD is a five-point scale ranging from 0 (Clear) to 4 (Severe) assessing inflammatory signs of atopic dermatitis. The demographic and disease characteristics of patients in the OLE trial ≥6 years old are detailed in Table 13 below.

TABLE 13
Demographic & Disease Characteristics at Trial Commencement

| | | 0.15% cream to 0.15% | | Vehicle to 0.15% | |
|---|---|---|---|---|---|
| | | cream (n = 439) | | cream (n = 218) | |
| Age in years, mean (SD) | | 19.4 | (16.4) | 20.5 | (17.9) |
| Female at birth, n (%) | | 244 | (55.6) | 122 | (56.0) |
| Not Hispanic or Latino, n (%) | | 361 | (82.2) | 182 | (83.5) |
| Race, n (%) | White | 272 | (62.0) | 139 | (63.8) |
| | Asian | 63 | (14.4) | 35 | (16.1) |
| | Black or African- | 58 | (13.2) | 31 | (14.2) |
| | American | | | | |
| | American-Indian or | 6 | (1.4) | 0 | |
| | Alaskan Native | | | | |
| | Native Hawaiian, Other | 1 | (0.2) | 0 | |
| | Pacific Islander | | | | |
| | More than one race | 20 | (4.6) | 7 | (3.2) |
| | Other | 19 | (4.3) | 6 | (2.8) |
| Fitzpatrick Skin | I to III | 245 | (55.8) | 120 | (55.1) |
| Type at Screening, n | IV to VI | 194 | (44.2) | 98 | (45.0) |
| (%) | | | | | |
| Baseline vIGA-AD, | 2 (mild) | 165 | (37.6) | 78 | (35.8) |
| n (%) | 3 (moderate) | 98 | (22.3) | 88 | (40.4) |

| | | | |
|---|---|---|---|
| Disease characteristics, | EASI | 4.4 (2.4) | 8.0 (5.5) |
| mean (median) [range] | | [0.0-45.4] | [0.0-44.0] |
| | BSA | 7.2 (3.5) | 12.9 (8.0) |
| | | [0.0-88.0] | [0.0-87.0] |
| | WI-NRS | 3.2 (3) | 4.2 (4) |
| | | [0-10] | [0-10] |

BSA: body surface area; EASI: Eczema Area and Severity Index; SD: standard deviation; vIGA-AD: Validated Investigator Global Assessment for Atopic Dermatitis; WI-NRS: Worst Itch Numeric Rating Scale.

The secondary efficacy endpoints were: (1) vIGA-AD value 0 or 1 at each assessment; (2) vIGA-AD success (defined as vIGA-AD value of 0 or 1 plus a 2-grade improvement from baseline); (3) WI-NRS score over time; and (4) EASI score over time.

No new safety signals were observed over up to 56 weeks of treatment. 96.3% of patients who experienced treatment-emergent adverse events (TEAEs) had AEs of mild or moderate severity. At each visit, ≥98.1% of patients showed no evidence of irritation on investigator assessment of local tolerability. For patient-rated local tolerability, ≥90.5% of patients reported no or mild sensation at each visit. The AEs leading to discontinuation were: atopic dermatitis (n=5), nausea (n=2), application site dermatitis (n=2), other preferred term (n=11; 1 patient per preferred term). The safety population was defined as all patients who enrolled and received ≥1 confirmed dose of trial medication.

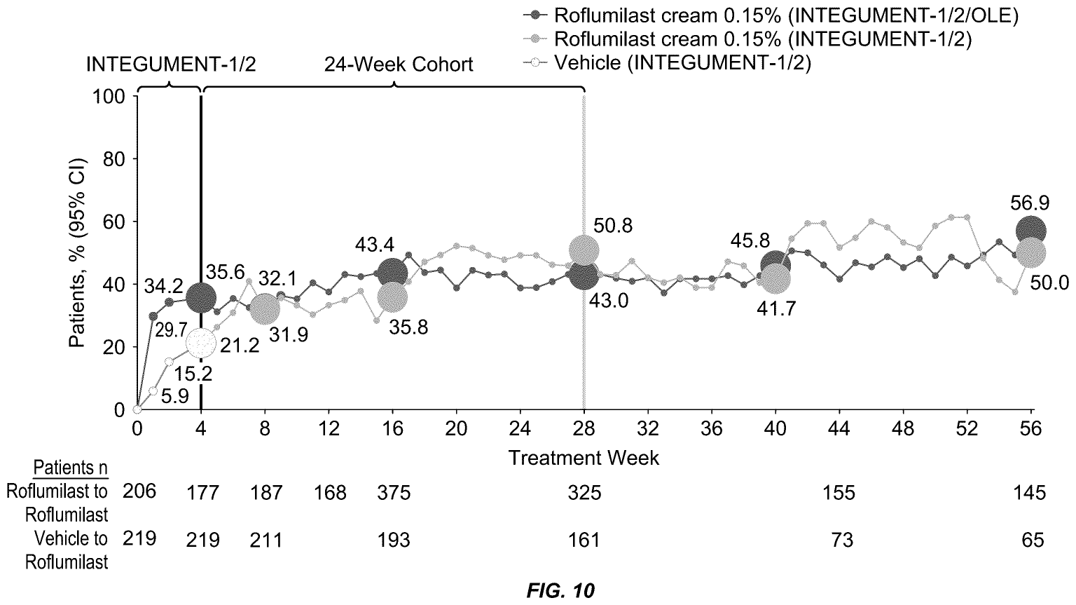

The results of the studies are set forth in FIG. 10, which provides a line plot of the proportion of all patients in the study achieving ≥4-point improvement from patient trial baseline in WI-NRS over the course of the 56 weeks. WI-NRS is an 11-point scale ranging from 0 [no itch] to 10 [worst itch imaginable]. Among patients aged ≥12 years with baseline WI-NRS ≥4 at week 56 of the study, 56.9% of participants who had rolled over from previous treatment with roflumilast cream 0.15% and 50.0% of participants who had switched to once-daily roflumilast cream 0.15% treatment achieved a significant reduction (≥4-point) in itch based on daily WI-NRS. Summaries of vIGA-AD (Table 14) and EASI (Table 15) over time throughout this study are presented below.

TABLE 14
vIGA-AD Scores Over Time

| | | | Vehicle/ | Pooled |
|---|---|---|---|---|
| | | Roflumilast Cream | Roflumilast Cream | Roflumilast Cream |
| Visit | | 0.05% or 0.15% | 0.05% or 0.15% | 0.05% or 0.15% |
| Result/Endpoint | Statistic | (N = 382) | (N = 180) | (N = 562) |

Primary Baseline - Observed

| | | | | | | | |
|---|---|---|---|---|---|---|---|
| 0 = Clear | n (%) | 0 | | 7 | (3.9) | 7 | (1.2) |
| 1 = Almost Clear | n (%) | 0 | | 20 | (11.1) | 20 | (3.6) |
| 2 = Mild | n (%) | 86 | (22.5) | 78 | (43.3) | 164 | (29.2) |
| 3 = Moderate | n (%) | 296 | (77.5) | 71 | (39.4) | 367 | (65.3) |
| 4 = Severe | n (%) | 0 | | 4 | (2.2) | 4 | (0.7) |

OLE Week 4 - Observed

| | | | | | | | |
|---|---|---|---|---|---|---|---|
| 0 = Clear | n (%) | 35 | (9.2) | 20 | (11.1) | 55 | (9.8) |
| 1 = Almost Clear | n (%) | 103 | (27.0) | 53 | (29.4) | 156 | (27.8) |
| 2 = Mild | n (%) | 143 | (37.4) | 63 | (35.0) | 206 | (36.7) |
| 3 = Moderate | n (%) | 76 | (19.9) | 38 | (21.1) | 114 | (20.3) |
| 4 = Severe | n (%) | 3 | (0.8) | 1 | (0.6) | 4 | (0.7) |
| Missing | n (%) | 22 | (5.8) | 5 | (2.8) | 27 | (4.8) |

OLE Week 12 - Observed

| | | | | | | | |
|---|---|---|---|---|---|---|---|
| 0 = Clear | n (%) | 50 | (13.1) | 31 | (17.20) | 81 | (14.4) |
| 1 = Almost Clear | n (%) | 93 | (24.3) | 58 | (32.20) | 151 | (26.9) |
| 2 = Mild | n (%) | 122 | (31.9) | 43 | (23.90) | 165 | (29.4) |
| 3 = Moderate | n (%) | 65 | (17.0) | 27 | (15.00) | 92 | (16.4) |
| 4 = Severe | n (%) | 4 | (1.0) | 2 | (1.10) | 6 | (1.1) |
| Missing | n (%) | 48 | (12.6) | 19 | (10.60) | 67 | (11.9) |

OLE Week 36 - Observed

| | | | | | | | |
|---|---|---|---|---|---|---|---|
| 0 = Clear | n (%) | 60 | (15.90) | 30 | (17.00) | 90 | (16.3) |
| 1 = Almost Clear | n (%) | 76 | (20.20) | 40 | (22.70) | 116 | (21.0) |
| 2 = Mild | n (%) | 86 | (22.80) | 47 | (26.70) | 133 | (24.1) |
| 3 = Moderate | n (%) | 42 | (11.10) | 15 | (8.50) | 57 | (10.3) |
| 4 = Severe | n (%) | 0 | | 0 | | 0 | |
| Missing | n (%) | 113 | (30.00) | 44 | (25.00) | 157 | (28.4) |

OLE Week 52 - Observed

| | | | | | | | |
|---|---|---|---|---|---|---|---|
| 0 = Clear | n (%) | 75 | (19.90) | 39 | (22.20) | 114 | (20.6) |
| 1 = Almost Clear | n (%) | 81 | (21.50) | 39 | (22.20) | 120 | (21.7) |
| 2 = Mild | n (%) | 71 | (18.80) | 30 | (17.00) | 101 | (18.3) |
| 3 = Moderate | n (%) | 25 | (6.60) | 12 | (6.80) | 37 | (6.7) |
| 4 = Severe | n (%) | 1 | (0.30) | 1 | (0.60) | 2 | (0.4) |
| Missing | n (%) | 124 | (32.90) | 55 | (31.30) | 179 | (32.4) |

TABLE 15
EASI Scores Over Time

| | | | Vehicle/ | Pooled |
|---|---|---|---|---|
| | | Roflumilast Cream | Roflumilast Cream | Roflumilast Cream |
| Visit | | 0.05% or 0.15% | 0.05% or 0.15% | 0.05% or 0.15% |
| Endpoint Result | Statistic | (N = 382) | (N = 180) | (N = 562) |

OLE Week 4

| | | | | | | | |
|---|---|---|---|---|---|---|---|
| EASI-50 | | | | | | | |
| Yes | n (%) | 255 | (70.80) | 85 | (50.60) | 340 | (64.40) |
| No | n (%) | 105 | (29.20) | 83 | (49.40) | 188 | (35.60) |
| EASI-75 | | | | | | | |
| Yes | n (%) | 166 | (46.10) | 52 | (31.00) | 218 | (41.30) |
| No | n (%) | 194 | (53.90) | 116 | (69.00) | 310 | (58.70) |
| EASI-90 | | | | | | | |
| Yes | n (%) | 84 | (23.30) | 27 | (16.10) | 111 | (21.00) |
| No | n (%) | 276 | (76.70) | 141 | (83.90) | 417 | (79.00) |
| EASI-100 | | | | | | | |
| Yes | n (%) | 33 | (9.20) | 17 | (10.10) | 50 | (9.50) |
| No | n (%) | 327 | (90.80) | 151 | (89.90) | 478 | (90.50) |

OLE Week 12

| | | | | | | | |
|---|---|---|---|---|---|---|---|
| EASI-50 | | | | | | | |
| Yes | n (%) | 247 | (74.00) | 97 | (62.60) | 344 | (70.30) |
| No | n (%) | 87 | (26.00) | 58 | (37.40) | 145 | (29.70) |
| EASI-75 | | | | | | | |
| Yes | n (%) | 181 | (54.20) | 68 | (43.90) | 249 | (50.90) |
| No | n (%) | 153 | (45.80) | 87 | (56.10) | 240 | (49.10) |
| EASI-90 | | | | | | | |
| Yes | n (%) | 95 | (28.40) | 37 | (23.90) | 132 | (27.00) |
| No | n (%) | 239 | (71.60) | 118 | (76.10) | 357 | (73.00) |
| EASI-100 | | | | | | | |
| Yes | n (%) | 48 | (14.40) | 24 | (15.50) | 72 | (14.70) |
| No | n (%) | 286 | (85.60) | 131 | (84.50) | 417 | (85.30) |

OLE Week 24

| | | | | | | | |
|---|---|---|---|---|---|---|---|
| EASI-50 | | | | | | | |
| Yes | n (%) | 244 | (82.20) | 101 | (73.70) | 345 | (79.50) |
| No | n (%) | 53 | (17.80) | 36 | (26.30) | 89 | (20.50) |
| EASI-75 | | | | | | | |
| Yes | n (%) | 183 | (61.60) | 82 | (59.90) | 265 | (61.10) |
| No | n (%) | 114 | (38.40) | 55 | (40.10) | 169 | (38.90) |
| EASI-90 | | | | | | | |
| Yes | n (%) | 109 | (36.70) | 43 | (31.40) | 152 | (35.00) |
| No | n (%) | 188 | (63.30) | 94 | (68.60) | 282 | (65.00) |
| EASI-100 | | | | | | | |
| Yes | n (%) | 59 | (19.90) | 30 | (21.90) | 89 | (20.50) |
| No | n (%) | 238 | (80.10) | 107 | (78.10) | 345 | (79.50) |

OLE Week 36

| | | | | | | | |
|---|---|---|---|---|---|---|---|
| EASI-50 | | | | | | | |
| Yes | n (%) | 214 | (81.10) | 93 | (73.80) | 307 | (78.70) |
| No | n (%) | 50 | (18.90) | 33 | (26.20) | 83 | (21.30) |
| EASI-75 | | | | | | | |
| Yes | n (%) | 163 | (61.70) | 66 | (52.40) | 229 | (58.70) |
| No | n (%) | 101 | (38.30) | 60 | (47.60) | 161 | (41.30) |
| EASI-90 | | | | | | | |
| Yes | n (%) | 103 | (39.00) | 42 | (33.30) | 145 | (37.20) |
| No | n (%) | 161 | (61.00) | 84 | (66.70) | 245 | (62.80) |
| EASI-100 | | | | | | | |
| Yes | n (%) | 58 | (22.00) | 28 | (22.20) | 86 | (22.10) |
| No | n (%) | 206 | (78.00) | 98 | (77.80) | 304 | (77.90) |

OLE Week 52

| | | | | | | | |
|---|---|---|---|---|---|---|---|
| EASI-50 | | | | | | | |
| Yes | n (%) | 220 | (87.00) | 82 | (71.30) | 302 | (82.10) |
| No | n (%) | 33 | (13.00) | 33 | (28.70) | 66 | (17.90) |
| EASI-75 | | | | | | | |
| Yes | n (%) | 182 | (71.90) | 67 | (58.30) | 249 | (67.70) |
| No | n (%) | 71 | (28.10) | 48 | (41.70) | 119 | (32.30) |
| EASI-90 | | | | | | | |
| Yes | n (%) | 121 | (47.80) | 43 | (37.40) | 164 | (44.60) |
| No | n (%) | 132 | (52.20) | 72 | (62.60) | 204 | (55.40) |
| EASI-100 | | | | | | | |
| Yes | n (%) | 74 | (29.20) | 35 | (30.40) | 109 | (29.60) |
| No | n (%) | 179 | (70.80) | 80 | (69.60) | 259 | (70.40) |

The trial overall showed a median duration 281 days (95% confidence interval) of disease control (defined as the duration of vIGA-AD of 0/1 with adequate control of signs and symptoms of AD on BIW dosing following achievement of vIGA-AD of 0), among the 19.8% (n=130) of patients who achieved vIGA-AD of 0 and switched to proactive BIW dosing. 75 of these 130 patients (57.7%) maintained “disease control” through their final visit.

It will be understood that all embodiments described herein may be applied to all aspects of the invention and vice versa. Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The following studies and protocols illustrate embodiments of the methods described herein.

Any of the above protocols or similar variants thereof can be described in various documentation associated with a pharmaceutical product. This documentation can include, without limitation, protocols, statistical analysis plans, investigator brochures, clinical guidelines, medication guides, risk evaluation and mediation programs, prescribing information and other documentation that may be associated with a pharmaceutical product. It is specifically contemplated that such documentation may be physically packaged with a pharmaceutical product according to the present disclosure as a kit, as may be beneficial or as set forth by regulatory authorities.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

What is claimed is:

1. A method of treating a patient suffering from atopic dermatitis, the method comprising:
topically administering to the patient a pharmaceutical composition comprising 0.05% to 0.30% roflumilast once daily,
wherein the pharmaceutical composition reduces itch as measured by the Worst Itch Numerical Rating Scale (WI-NRS) in the patient within one week following said topical administration.

2. The method of claim 1, wherein the pharmaceutical composition comprises 0.05% roflumilast.

3. The method of claim 1, wherein the pharmaceutical composition comprises 0.15% roflumilast.

4. The method of claim 1, wherein the pharmaceutical composition is an emulsion comprising a solvent, water, an emulsifier, and a moisturizer.

5. The method of claim 1, wherein the pharmaceutical composition is a cream.

6. The method of claim 5, wherein the pharmaceutical composition comprises diethylene glycol monoethyl ether.

7. The method of claim 5, wherein the pharmaceutical composition comprises an emulsifier blend comprising cetearyl alcohol, dicetyl phosphate and ceteth-10 phosphate.

8. The method of claim 4, wherein the pharmaceutical composition comprises an emulsifier blend comprising cetearyl alcohol, dicetyl phosphate and ceteth-10 phosphate.

9. The method of claim 7, wherein the pharmaceutical composition reduces itch as measured by the WI-NRS by four-points relative to baseline within one week following said topical administration.

10. A method of treating a patient suffering from atopic dermatitis, the method comprising:
topically administering to the patient a pharmaceutical composition comprising 0.05% to 0.30% roflumilast once daily,
wherein the pharmaceutical composition reduces itch as measured by the WI-NRS in the patient within 24 hours following said topical administration.

11. The method of claim 10, wherein the pharmaceutical composition comprises 0.05% roflumilast.

12. The method of claim 10, wherein the pharmaceutical composition comprises 0.15% roflumilast.

13. The method of claim 10, wherein the pharmaceutical composition is an emulsion comprising a solvent, water, an emulsifier, and a moisturizer.

14. The method of claim 10, wherein the pharmaceutical composition is a cream.

15. The method of claim 14, wherein the pharmaceutical composition comprises diethylene glycol monoethyl ether.

16. The method of claim 14, wherein the pharmaceutical composition comprises an emulsifier blend comprising cetearyl alcohol, dicetyl phosphate and ceteth-10 phosphate.

17. The method of claim 15, wherein the pharmaceutical composition comprises an emulsifier blend comprising cetearyl alcohol, dicetyl phosphate and ceteth-10 phosphate.

18. The method of claim 16, wherein the pharmaceutical composition reduces itch as measured by the Worst Itch Numerical Rating Scale (WI-NRS) by four-points relative to baseline within 24 hours following said topical administration.

19. A method of treating a patient suffering from atopic dermatitis, the method comprising:
topically administering to the patient a pharmaceutical composition comprising 0.05% to 0.30% roflumilast once daily,
wherein the pharmaceutical composition reduces itch as measured by the WI-NRS in the patient within 48 hours following said topical administration.

20. The method of claim 19, wherein the pharmaceutical composition comprises 0.05% roflumilast.

21. The method of claim 19, wherein the pharmaceutical composition comprises 0.15% roflumilast.

22. The method of claim 19, wherein the pharmaceutical composition is an emulsion comprising a solvent, water, an emulsifier, and a moisturizer.

23. The method of claim 19, wherein the pharmaceutical composition is a cream.

24. The method of claim 23, wherein the pharmaceutical composition comprises diethylene glycol monoethyl ether.

25. The method of claim 23, wherein the pharmaceutical composition comprises an emulsifier blend comprising cetearyl alcohol, dicetyl phosphate and ceteth-10 phosphate.

26. The method of claim 24, wherein the pharmaceutical composition comprises an emulsifier blend comprising cetearyl alcohol, dicetyl phosphate and ceteth-10 phosphate.

27. The method of claim 25, wherein the pharmaceutical composition reduces itch as measured by the Worst Itch Numerical Rating Scale (WI-NRS) by four-points relative to baseline within 24 hours following said topical administration.
